(19) United States
(12) Patent Application Publication
(10) Pub. No.: US 2025/0090871 A1
(43) Pub. Date: Mar. 20, 2025

(54) SIMULATION SOFTWARE AND TOOLS FOR EVALUATING HISTOTRIPSY THERAPY FOR A GIVEN POSE AND POSITION OF A THERAPY ARRAY

(71) Applicant: HistoSonics, Inc., Plymouth, MN (US)

(72) Inventors: John SNELL, Charlottesville, VA (US); Jon David SCHELL, Shoreview, MN (US); Jonathan Matthew CANNATA, Ann Arbor, MI (US)

(21) Appl. No.: 18/886,807

(22) Filed: Sep. 16, 2024

Related U.S. Application Data
(60) Provisional application No. 63/583,086, filed on Sep. 15, 2023.

(51) Int. Cl. A61N 7/00; G16H 20/40

(52) U.S. Cl. CPC A61N 7/00; G16H 20/40

(57) ABSTRACT

Simulation software and planning tools are provided that can simulate treatment, allowing a user to identify preferred poses or positions of a histotripsy therapy transducer array for a given treatment location. The planning tools can identify obstructed transducer elements for a given pose/position, and can provide pressure simulations to estimate driving voltages required to generate cavitation at the treatment location. A digital treatment plan made with the planning tool can transferred to a histotripsy system for use during the therapy procedure. A histotripsy therapy system configured for the treatment of tissue is provided, which may implement the digital treatment plan from the simulation software and planning tools.

### PRIORITY CLAIM

This patent application claims priority to U.S. Provisional Application No. 63/583,086, titled “HISTOTRIPSY SYSTEMS AND METHODS” and filed on Sep. 15, 2023, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU) cryo or radiation, Histotripsy relies on the mechanical action of cavitation for tissue destruction and not on heat, cold or ionizing energy.

### SUMMARY OF THE DISCLOSURE

A method of planning histotripsy therapy is provided, comprising obtaining at least one diagnostic image of a subject including a target tissue location; generating a three-dimensional (3D) digital model of the subject and the target tissue location from the at least one diagnostic image; positioning a virtual histotripsy therapy transducer array near the 3D model with a geometric focus of the virtual histotripsy therapy transducer positioned on or near the target tissue location in the 3D digital model; sampling the virtual histotripsy therapy transducer array to provide starting points for a plurality of rays that correspond to simulated ultrasound energy delivered by transducer elements of the virtual histotripsy therapy transducer; tracing the plurality of rays through the 3D digital model; and identifying one or more obstructed or partially obstructed transducer elements of the virtual histotripsy therapy transducer array corresponding to rays that pass through an obstruction in the 3D digital model.

In one aspect, the method further comprises displaying the rays that pass through an obstruction to a user.

In some aspects, the method comprises providing a first graphical representation of the rays that pass through an obstruction and providing a second graphical representation of the rays that do not pass through an obstruction.

In some aspects, rays that pass through an obstruction are displayed in a first color and rays that do not pass through an obstruction are displayed in a second color.

In one aspect, the 3D digital model further comprises a skin surface model of the subject.

In some aspects, the method comprises presenting a map of rays that pass through an obstruction on the skin surface model.

In some aspects, displaying the rays that pass through an obstruction further comprises providing a 2D view of the plurality of rays.

In another aspect, the method further comprises: receiving an input to adjust a position or pose of the virtual histotripsy therapy transducer; re-sampling the virtual histotripsy therapy transducer array to provide starting points for a second plurality of rays that correspond to simulated ultrasound energy delivered by transducer elements of the virtual histotripsy therapy transducer array; tracing the second plurality of rays through the 3D digital model; and identifying one or more obstructed or partially obstructed transducer elements of the virtual histotripsy therapy transducer array corresponding to rays that pass through an obstruction in the 3D digital model.

In some aspects, the method further comprises generating a digital treatment plan that includes the target tissue location and a pose or position of the virtual histotripsy therapy transducer array.

In one aspect, the digital treatment plan further includes a depth and size of the target tissue location.

In another aspect, the method includes segmenting one or more target tissues or organs in proximity to the target tissue location.

A method of planning histotripsy therapy is provided, comprising: obtaining at least one diagnostic image of a subject including a target tissue location; generating a three-dimensional (3D) digital model of the subject and the target tissue location from the at least one diagnostic image; positioning a virtual histotripsy therapy transducer array near the subject with a geometric focus of the histotripsy therapy transducer positioned on or near the target tissue location; sampling the virtual histotripsy therapy transducer array to provide starting points for a plurality of rays that correspond to ultrasound energy delivered by transducer elements of the histotripsy therapy transducer array; tracing the plurality of rays through a tissue boundary in the 3D digital model; and identifying a refraction angle of one or more rays that pass from a first speed of sound medium through the tissue boundary into a second speed of sound medium.

In another aspect, the method includes indicating the refraction angle of the rays to a user.

In one aspect, the method includes displaying the refraction angle of the rays to the user.

In some aspects, the method includes displaying an expected focus of the rays based on the refraction angle.

In other aspects, the method includes displaying a skin surface model of the subject that includes a map of the rays that are affected by the refraction angle.

In one aspect, the method includes providing a 2D view of the rays.

In some aspects, the method comprises dividing a path length of each ray into a plurality of segments with known speeds of sound.

In other aspects, the method comprises calculating a time delay for each of the rays to arrive at the geometric focus.

In some aspects, the method comprises adjusting the timing of ultrasound delivery for a plurality of transducer elements of the histotripsy therapy transducer based on the calculated time delays.

A method of planning histotripsy therapy, comprising: obtaining at least one diagnostic image of a subject including a target tissue location; generating a three-dimensional (3D) digital model of the subject and the target tissue location from the at least one diagnostic image; positioning a virtual histotripsy therapy transducer array near the subject with a geometric focus of the histotripsy therapy transducer array positioned on or near the target tissue location; sampling the histotripsy therapy transducer to provide starting points for a plurality of rays that correspond to ultrasound energy delivered by transducer elements of the histotripsy therapy transducer array; tracing the plurality of rays through the 3D digital model; generating a pressure simulation within 3D digital model based on the plurality of rays; and estimating one or more driving voltages of the virtual histotripsy therapy transducer array required to generate cavitation at the target tissue location based on the pressure simulation.

In some aspects, generating the pressure simulation further comprises computing a Rayleigh-Sommerfeld integral over a 2D or 3D computational domain.

In one aspect, the method comprises displaying rays that pass through an obstruction to a user.

In another aspect, the method comprises providing a first graphical representation of rays that pass through an obstruction and providing a second graphical representation of rays that do not pass through an obstruction.

In some aspects, rays that pass through an obstruction are displayed in a first color and rays that do not pass through an obstruction are displayed in a second color.

In some aspects, the 3D digital model further comprises a skin surface model of the subject.

In another aspect, the method comprises presenting a map of rays that pass through an obstruction on the skin surface model.

In some aspects, displaying the rays that pass through an obstruction further comprises providing a 2D view of the plurality of rays.

In another aspect, the method comprises: receiving an input to adjust a position or pose of the virtual histotripsy therapy transducer; re-sampling the virtual histotripsy therapy transducer array to provide starting points for a second plurality of rays that correspond to simulated ultrasound energy delivered by transducer elements of the virtual histotripsy therapy transducer array; tracing the second plurality of rays through the 3D digital model; and identifying one or more obstructed or partially obstructed transducer elements of the virtual histotripsy therapy transducer array corresponding to rays that pass through an obstruction in the 3D digital model.

In some aspects, the method includes generating a digital treatment plan that includes the target tissue location and a pose or position of the virtual histotripsy therapy transducer array.

In another aspect, the digital treatment plan further includes a depth and size of the target tissue location.

In some aspects, the method includes segmenting one or more target tissues or organs in proximity to the target tissue location.

An ultrasound system is provided, comprising: a robotic positioning system; an ultrasound transducer array disposed on the robotic positioning system, the ultrasound transducer array being configured to deliver ultrasound pulses into a subject; a diagnostic imaging system configured to obtain pre-procedural images of the subject; a display; one or more processors operatively coupled to the robotic positioning system, the diagnostic imaging system, the ultrasound transducer array, and the display; a non-transitory computing device readable medium having instructions stored thereon for generating a treatment plan for ultrasound therapy, wherein the instructions are executable by the one or more processors to cause the ultrasound system to: obtain at least one diagnostic image of the subject including a target tissue location with the diagnostic imaging system; generate a three-dimensional (3D) digital model of the subject and the target tissue location from the at least one diagnostic image; position the ultrasound transducer array near the subject with a geometric focus of the histotripsy therapy transducer positioned on or near the target tissue location; sample the ultrasound transducer array to provide starting points for a plurality of rays that correspond to ultrasound energy delivered by transducer elements of the ultrasound transducer array; trace the plurality of rays through the 3D digital model; identify one or more obstructed rays corresponding to rays that pass through an obstruction in the subject; and display the plurality of rays and the one or more obstructed rays overlaid on the 3D digital model on the display.

In some aspects, obstructed rays are displayed in a first color and unobstructed rays are displayed in a second color.

In other aspects, the instructions are executable by the one or more processors to cause the ultrasound system to display a 3D model of the skin surface of the subject that includes a map of the obstructed rays.

In some aspects, the instructions are executable by the one or more processors to cause the ultrasound system to provide a 2D view of the obstructed rays.

A histotripsy treatment planning tool is also provided, comprising: a display; a user interface; one or more processors operatively coupled to the display and the user interface; a non-transitory computing device readable medium having instructions stored thereon for generating a treatment plan for ultrasound therapy, wherein the instructions are executable by the one or more processors to: receive one or more diagnostic images of a subject including a target tissue location; present a three-dimensional (3D) model of the subject and the target tissue location on the display; present a virtual ultrasound therapy transducer array in proximity to the 3D model of the subject; receive an input from the user-interface that includes instructions to modify a pose or position of the virtual ultrasound therapy transducer array; simulate a plurality of rays from the virtual ultrasound therapy transducer array for a given pose and position into the 3D model of the subject and into the target tissue location; and present information on the display indicating if any of the plurality of rays pass through an obstruction within the 3D model of the subject.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2 is one embodiment of a histotripsy therapy and imaging system with a coupling system.

FIG. 3 is one example of an ultrasound pulse for generating histotripsy via a shock scattering mechanism.

FIGS. 4A-4M and FIG. 5 illustrate user interface workflow screens for a treatment planning tool.

FIGS. 6A-6B illustrate an example of ray tracing being used with a histotripsy therapy system to identify potential obstructions between the therapy transducer and the focus.

FIGS. 7A-7G are examples of a treatment planning tool that can use ray tracing to provide information to a user regarding potential obstructions and optimal transducer placement.

FIGS. 8A-8F illustrate a treatment planning tool that provides information to a user regarding potential obstructions, optimal transducer placement, and potential collisions between the transducer, coupling container, and/or patient.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system may include various sub-systems, including a Cart, Therapy, Integrated Imaging, Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

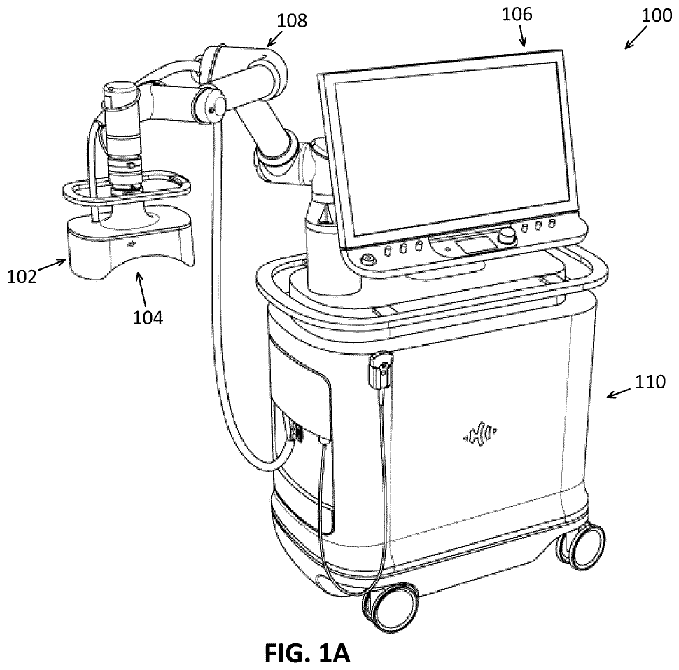

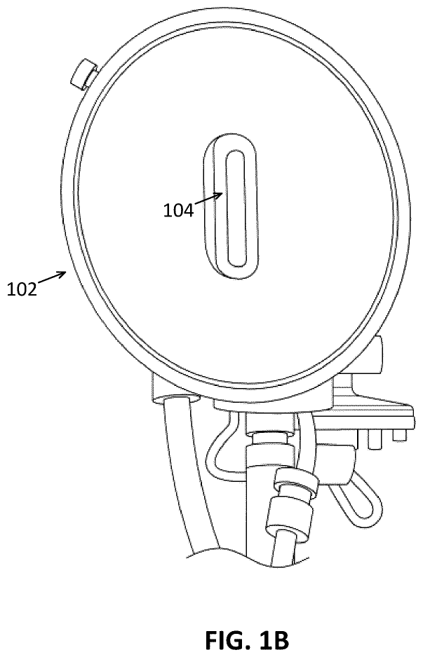

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described above, the histotripsy system may include integrated imaging. However, in other embodiments, the histotripsy system can be configured to interface with separate imaging systems, such as C-arm, fluoroscope, cone beam CT, MRI, etc., to provide real-time imaging during histotripsy therapy. In some embodiments, the histotripsy system can be sized and configured to fit within a C-arm, fluoroscope, cone beam CT, MRI, etc.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses typically with a 1-2 cycles of high amplitude negative/tensile phase pressure exceeding the intrinsic threshold to generate cavitation in the medium (e.g., ˜24-28 MPa for water-based soft tissue), 2) Shock-Scattering Histotripsy: Delivers typically pulses 1-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

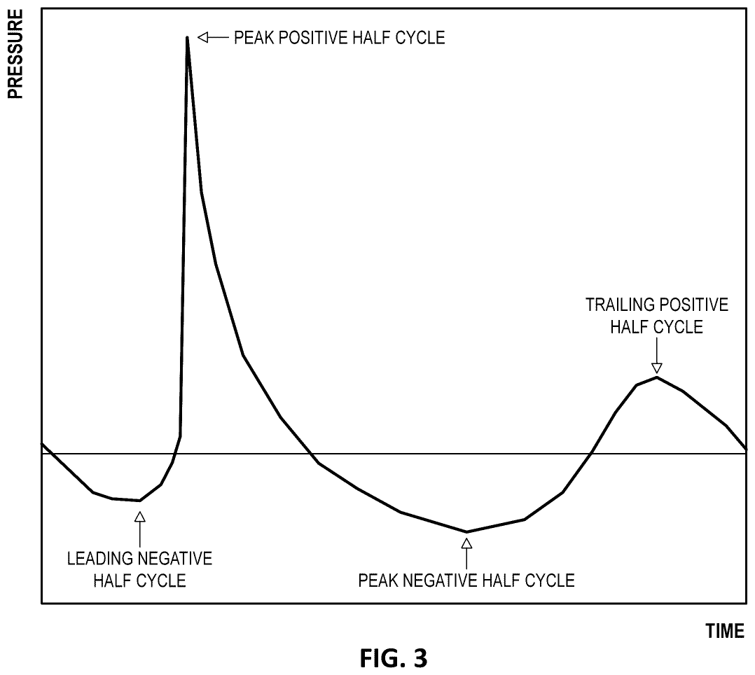

FIG. 3 illustrates an ultrasound pulse that can be used for shock scattering histotripsy. As shown the ultrasound pulse can include a leading negative half cycle, a peak positive half cycle, a peak negative half cycle, and a trailing peak positive half cycle (with the pulse traveling from right to left on the page). As shown, the trailing peak positive cycle has a lower amplitude than the peak positive cycle. This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface if the amplitude of those cycles is sufficient, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When the amplitude(s) of positive half cycle(s) of each pulse are limited, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue.

In order to create and deliver histotripsy and derivatives of histotripsy, the therapy sub-system may also comprise components, including but not limited to, one or more function generators, amplifiers, therapy transducers and power supplies.

The therapy transducer can comprise a single element or multiple elements configured to be excited with high amplitude electric pulses (>1000V or any other voltage that can cause harm to living organisms). The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezo-electric material. Transducers therefore operating at a high frequency require lower voltage to produce a given surface pressure than is required by low frequency therapy transducers. In some embodiments, the transducer elements are formed using a piezoelectric-polymer composite material or a solid piezoelectric material. Further, the piezoelectric material can be of polycrystalline/ceramic or single crystalline formulation. In some embodiments the transducer elements can be formed using silicon using MEMs technology, including CMUT and PMUT designs.

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other systems may include other acoustic cavitation/histotripsy, HIFU, HITU, radiation therapy, radiofrequency, microwave, and cryoablation systems, navigation and localization systems, open surgical, laparoscopic, single incision/single port, endoscopic and non-invasive surgical robots, laparoscopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor L1 in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the piezoelectric element. For example, because a piezoelectric clement operating at 1 MHz transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation. Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparoscopic towers, vision systems, endoscope systems and towers, ultrasound enabled endoscopic ultrasound (flexible and rigid), percutaneous/endoscopic/laparoscopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparoscopic surgical robot). In many of these environments, a therapy transducer may be utilized at or around the time of use, for example, of an optically guided endoscope/bronchoscope, or as another example, at the time a laparoscopic robot (e.g., Intuitive Da Vinci* Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparoscopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

They system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in US Patent Pub. No. 2013/0255426 to Kassow et al. which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable-distance controlled positions.

Tracking may be configured to comprise time-controlled tracking and/or distance-controlled tracking.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or de-activation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degree of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparoscopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the robotic arm may comprise a robotic arm on a laparoscopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparoscopic arms (and instruments) and various histotripsy system components. For example, a laparoscopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparoscopic camera. In other related aspects, a similar approach may be utilized with a combination of an endoscopic and non-invasive approach, and further, with a combination of an endoscopic, laparoscopic and non-invasive approach.

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles. Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below). Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparoscopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Coupling

Systems may comprise a variety of Coupling sub-system embodiments, of which are enabled and configured to allow acoustic coupling to the patient to afford effective acoustic access for ultrasound visualization and acoustic cavitation/histotripsy (e.g., provide acoustic window and medium between the transducer(s) and patient, and support of). These may include different form factors of such, including open and enclosed device solutions, and some arrangements which may be configured to allow dynamic control over the acoustic medium (e.g., temperature, dissolved gas content, level of particulate filtration, sterility, volume, composition, etc.). Such dynamic control components may be directly integrated to the system (within the Cart), or may be in temporary/intermittent or continuous communication with the system, but externally situated in a separate device and/or cart.

The Coupling sub-system typically comprises, at a minimum, coupling medium (e.g., degassed water or water solutions), a reservoir/container to contain said coupling medium, and a support structure (including interfaces to other surfaces or devices). In most embodiments, the coupling medium is water, and wherein the water may be conditioned before or during the procedure (e.g., chilled, degassed, filtered, etc.). Various conditioning parameters may be employed based on the configuration of the system and its intended use/application.

The reservoir or medium container may be formed and shaped to various sizes and shapes, and to adapt/conform to the patient, allow the therapy transducer to engage/access and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. As a representative example, this may include a support frame. The container may be of various shapes, sizes, curvatures, and dimensions, and may be comprised of a variety of materials compositions (single, multiple, composites, etc.), of which may vary throughout. In some embodiments, it may comprise features such as films, drapes, membranes, bellows, etc. that may be insertable and removable, and/or fabricated within, of which may be used to conform to the patient and assist in confining/containing the medium within the container. It may further contain various sensors (e.g., volume/fill level), drains (e.g., inlet/outlet), lighting (e.g., LEDs), markings (e.g., fill lines, set up orientations, etc.), text (e.g., labeling), etc.

In one embodiment, the reservoir or medium container contains a scalable frame, of which a membrane and/or film may be positioned within, to afford a conformable means of contacting the reservoir (later comprising the treatment head/therapy transducer) as an interface to the patient, that further provides a barrier to the medium (e.g., water) between the patient and therapy transducer). In other embodiments, the membrane and/or film may comprise an opening, the patient contacting edge of which affords a fluid/mechanical seal to the patient, but in contrast allows medium communication directly with the patient (e.g., direct degassed water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers. The membrane form factor can be flat or pre-shaped prior to use. In other embodiments, the membrane could be inelastic (i.e., a convex shape) and pressed against the patient's skin to acoustically couple the transducer to the tissue. Systems and methods are further disclosed to control the level of contaminants (e.g., particulates, etc.) on the membrane to maintain the proper level of ultrasound coupling. Too many particulates or contaminants can cause scattering of the ultrasound waves. This can be achieved with removable films or coatings on the outer surfaces of the membrane to protect against contamination.

Said materials may be formed into useful membranes through molding, casting, spraying, ultrasonic spraying, extruding, and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded or over molded frame to provide mechanical stability to the membrane during handling including assembly, set up and take down of the coupling sub-system. Various parameters of the membrane can be optimized for this method of use, including thickness, thickness profile, density, formulation (e.g., polymer molecular weight and copolymer ratios, additives, plasticizers, etc.), including optimizing specifically to maximize acoustic transmission properties, including minimizing impact to cavitation initiation threshold values, and/or ultrasound imaging artifacts, including but not limited to membrane reflections, as representative examples.

Open reservoirs or medium containers may comprise various methods of filling, including using pre-prepared medium or water, that may be delivered into the containers, in some cases to a defined specification of water (level of temperature, gas saturation, etc.), or they may comprise additional features integral to the design that allow filling and draining (e.g., ports, valves, hoses, tubing, fittings, bags, pumps, etc.). These features may be further configured into or to interface to other devices, including for example, a fluidics system. In some cases, the fluidics system may be an in-house medium preparation system in a hospital or care setting room, or conversely, a mobile cart-based system which can prepare and transport medium to and from the cart to the medium container, etc.

Enclosed iterations of the reservoir or medium container may comprise various features for sealing, in some embodiments sealing to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing, pumping, filtering and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed, floor, etc.). In some embodiments, the support system comprises a mechanical arm with 3 or more degrees of freedom. Said arm may have a proximal interface with one or more locations (and features) of the bed, including but not limited to, the frame, rails, customized rails or inserts, as well as one or more distal locations of the reservoir or container. The arm may also be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or Coupling solution features (e.g., filling, draining, etc.). In some examples, the support structure robotic arm positional encoders may be used to coordinate the manipulation of the second arm (e.g. comprising the therapy transducer/treatment head), such as to position the therapy transducer to a desired/known location and pose within the coupling support structure.

Overall, significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

In addition, specific needs exist for enabling histotripsy delivery, including robotic histotripsy delivery, wherein one or more histotripsy therapy transducers may be configured to acoustically couple to a patient, using a completely sealed approach (e.g., no acoustic medium communication with the patient's skin) and allowing the one or more histotripsy transducers to be moved within the coupling solution without impeding the motion/movement of the robotic arm or interfering/disturbing the coupling interface, which could affect the intended treatment and/or target location.

Disclosed herein are histotripsy acoustic and patient coupling systems and methods, to enable histotripsy therapy/treatment, as envisioned in any setting, from interventional suite, operating room, hybrid suites, imaging centers, medical centers, office settings, mobile treatment centers, and/or others, as non-limiting examples. The following disclosure further describes novel systems used to create, control, maintain, modify/enhance, monitor and setup/takedown acoustic and patient coupling systems, in a variety of approaches, methods, environments, architectures and work-flows. In general, the disclosed novel systems may allow for a coupling medium, in some examples degassed water, to be interfaced between a histotripsy therapy transducer and a patient, wherein the acoustic medium provides sufficient acoustic coupling to said patient, allowing the delivery of histotripsy pulses through a user desired treatment location (and volume), where the delivery may require physically moving the histotripsy therapy transducer within a defined work-space comprising the coupling medium, and also where the coupling system is configured to allow said movement of the therapy transducer (and positioning system, e.g., robot) freely and unencumbered from by the coupling support system (e.g., a frame or manifold holding the coupling medium).

### Coupling System and Sub-Systems/Components

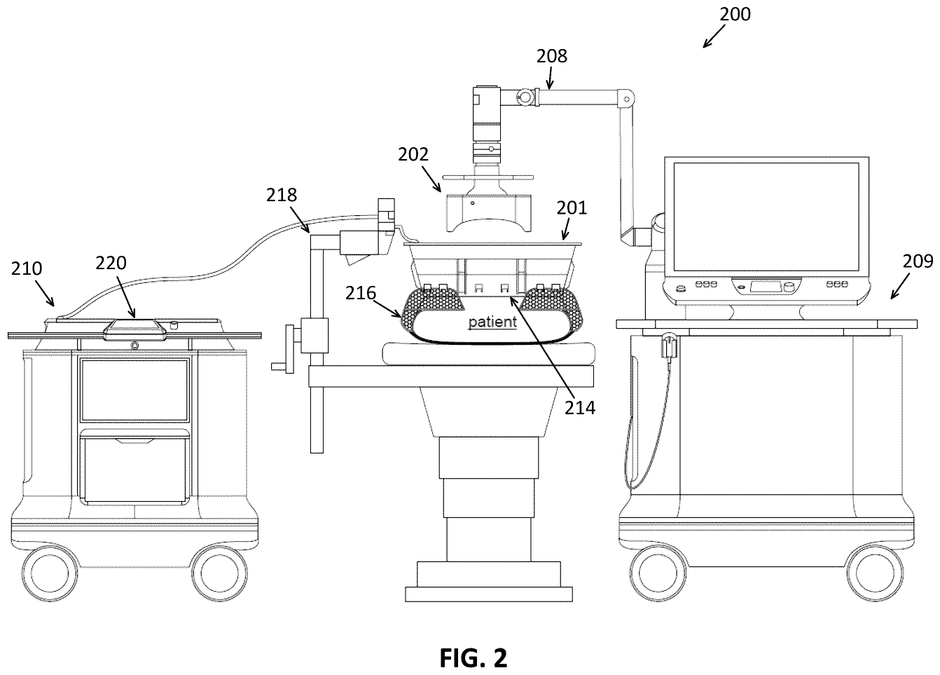

The disclosed histotripsy acoustic and patient coupling systems, in general, may comprise one or more of the following sub-systems and components, an example of which is depicted in FIG. 2, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

FIG. 2 illustrates one embodiment of a histotripsy therapy and imaging system 200, including a coupling assembly 201. As described above, a histotripsy therapy and imaging system can include a therapy transducer 202, an imaging system, a robotic positioning arm 208, and a fluidics cart 210. The robotic positioning arm may be attached to a therapy cart, such as cart 209.

The therapy and/or imaging transducers can be disposed within in the coupling assembly 201 which can further include a coupling membrane 214 and a membrane constraint 216 configured to prevent the membrane from expanding too far from the transducer. The coupling membrane can be filled with an acoustic coupling medium such as a fluid or a gel. The membrane constraint can be, for example, a semi-rigid or rigid material as compared to the membrane, and configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 201 is supported by a mechanical support arm 218 which can be load bearing in the x-y plane but allow for manual or automated z-axis adjustment. The mechanical support arm can be attached to the floor, the patient table, or the fluidics cart 210. The mechanical support is designed and configured to conform and hold the coupling membrane 214 in place against the patient's skin while still allowing movement of the therapy/imaging transducer relative to the patient and also relative to the coupling membrane 214 with the robotic positioning arm 208.

The fluidics cart 210 can include additional features, including a fluid tank 220, a cooling and degassing system, and a programmable control system. The fluidics cart is configured for external loading of the coupling membrane with automated control of fluidic sequences. Further details on the fluidics cart are provided below.

### Membranes/Barrier Films and Related Architectures

Membranes and barrier films may be composed of various biocompatible materials which allow conformal coupling to patient anatomy with minimal or no entrapped bubbles capable of interfering with ultrasound imaging and histotripsy therapy, and that are capable of providing a sealed barrier layer between said patient anatomy and the ultrasound medium, of which is contained within the work-space provided by the frame and assembly.

Membrane and barrier film materials may comprise flexible and elastomeric biocompatible materials/polymers, such as various thermoplastic and thermoset materials, as well as permanent or bioresorbable polymers. Additionally, the frame of the UMC can also comprise the same materials. In some examples, the membrane may be rigid or semi-rigid polymers which are pre-shaped or flat.

### Ultrasound Medium

As previously described, the ultrasound medium may comprise any applicable medium capable of providing sufficient and useful acoustic coupling to allow histotripsy treatments and enable sufficient clinical imaging (e.g., ultrasound). Ultrasound mediums, as a part of this disclosure and system, may comprise, but are not limited to, various aqueous solutions/mediums, including mixtures with other co-soluble fluids, of which may have preferred or more preferred acoustic qualities, including ability to match speed of sound, etc. Example mediums may comprise degassed water and/or mixtures/co-solutions of degassed water and various alcohols, such as ethanol.

### Mechanical Support Arms and Arm Architectures

In order to support the acoustic and patient coupling system, including providing efficient and ergonomic work-flows for users, various designs and configurations of mechanical support arms (and arm architectures) may be employed. Support arms may be configured with a range of degrees of freedom, including but not limited to allowing, x, y, z, pitch, roll and yaw, as well additional interfacing features that may allow additional height adjustment or translation.

Arms may comprise a varied number and type of joints and segments. Typically, arms may comprise a minimum of 2 segments. In some configurations, arms may comprise 3 to 5 segments.

Arms are also be configured to interface proximally to a main support base or base interface (e.g., robot, table, table/bed rail, cart, floor mount, etc.) and distally to the frame/assembly and overall “UMC” or “coupling solution”. This specific distal interface may further include features for controlling position/orientation of the frame/assembly, at the frame/assembly interface.

For example, in some embodiments, the arm/frame interface may comprise a ball joint wrist. In another example, the interface may include use of a gimbal wrist or an adjustable pitch and roll controlled wrist. These interfaces may be further employed with specific user interfaces and inputs, to assist with interacting with the various wrists, of which may include additional handles or knobs (as an unlimited example), to further enable positioning the UMC/coupling solution. For example, a gimbal wrist may benefit from allowing the frame/assembly to have 3 degrees of freedom (independent of the arm degrees of freedom), including pitch, roll and yaw adjustments.

Support arms, configured with arm wrists, further interfaced with frames/assemblies, may comprise features such as brakes, including cable or electronic actuated brakes, and quick releases, which may interact with one or more axis, individually, or in groupings. They may also include electronic lift systems and base supports. In some embodiments, these lift systems/base supports are co-located with robot arm bases, wherein said robot arm is equipped with the histotripsy therapy transducer configured to fit/work within the enclosed coupling solution. In other embodiments, the support arm is located on a separate cart. In some cases, the separate cart may comprise a fluidics system or user console. In other embodiments, it is interfaced to a bed/table, including but not limited to a rail, side surface, and/or bed/table base. In other examples/embodiments, it's interfaced to a floor-based structure/footing, capable of managing weight and tipping requirements.

### Fluidics Systems, Control Systems and System Architectures

As a part of overall fluidics management, histotripsy systems including acoustic/patient coupling systems, may be configured to include an automated fluidics system, which primarily is responsible for providing a reservoir for preparation and use of coupling medium, where preparation may include the ability to degas, chill, monitor, adjust, dispense/fill, and retrieve/drain coupling medium to/from the frame/assembly. The fluidics system may include an emergency high flow rate system for rapid draining of the coupling medium from the UMC. In some embodiments, the fluidics system can be configured for a single use of the coupling medium, or alternatively, for re-use of the medium. In some embodiments, the fluidics system can implement positive air pressure or vacuum to carry out leak tests of the UMC and membrane prior to filling with a coupling medium. Vacuum assist can also be used for removal of air from the UMC during the filling process. The fluidics system can further include filters configured to prevent particulate contamination from reaching the UMC.

The fluidics system may implemented in the form of a mobile fluidics cart. The cart may comprise an input tank, drain tank, degassing module, fill pump, drain pump, inert gas tank, air compressor, tubing/connectors/lines, electronic and manual controls systems and input devices, power supplies and one or more batteries. The cart in some cases may also comprise a system check vessel/reservoir for evaluating histotripsy system performance and related system diagnostics (configured to accommodate a required water volume and work-space for a therapy transducer).

### Histotripsy Treatment Planning Software Tools and Systems

Histotripsy therapy is provided by transmitting ultrasound signals from a plurality of transducer elements towards a common focal location. Pressures generated at this focal location induce cavitation in the target tissue to mechanically fractionate, liquefy, or lyse tissue. However, the efficiency and efficacy of therapy can be affected by a number of patient specific factors, including, for example, obstructions between the transducer array and the target tissue, variations in intervening tissue between the transducer and the focal location including tissue density changes, tissue type variations, and pockets of gas or air. Creating an optimal acoustic window to a particular target in the body can be quite challenging given a subject's anatomy and variations in body composition and makeup from subject to subject.

This disclosure provides treatment planning tools that can be implemented in software on one or more computing systems. In some embodiments, the treatment planning tools are implemented in a console of the histotripsy system. In other embodiments, the treatment planning tools are implemented on a cloud or remote server or computing platform. The cloud or remote computing platform can be in communication with the histotripsy system/console. For example, a physician or medical provider can create and review a treatment plan on a first computing platform (e.g., a personal computer, smartphone, tablet, etc.) and transfer or transmit the treatment plan to the histotripsy system console that will be used for the actual histotripsy procedure to carry out the treatment plan.

### Pre-Operative Imaging, Co-Registration, and Evaluation

Treatment planning tools are provided herein for Histotripsy therapy that can be used to plan and assist for histotripsy treatments and procedures. The treatment planning tools disclosed herein can import and co-register pre-operative imaging from a variety of imaging modalities (e.g., CT, CBCT, MRI, Ultrasound, X-ray, etc.) Software and hardware within the operating room, or remote to the operating room/treatment location, can provide treatment planning tools can be configured to allow a user to evaluate the pre-operative imaging and identify a tissue target such as a tumor. While the description herein refers to pre-operative imaging, it should be understood that in some embodiments, imaging can be performed during a histotripsy procedure, and any of the steps or techniques described herein can therefore apply to real-time or intra-operative imaging in addition to or instead of pre-operative imaging.

In some embodiments, the treatment planning tool can use imaging of the patient when the patient is lying on the procedure table or positioned on a scanner gantry, in the position that the patient will be in during the procedure. Additionally, any surgical prep equipment, gowns, clothing, and/or acoustic coupling containers that will be necessary for the procedure can be in place on or around the patient to provide the most accurate medical imaging of the patient's body for the planned procedure. For example, acoustic coupling containers as described herein and used with histotripsy procedures can be large rigid or semi-rigid containers that are placed on top of a patient and filled with an acoustic coupling medium (e.g., water) to allow for acoustic coupling of a therapy transducer array to the patient's skin. In some examples, these acoustic coupling containers can be filled with 30 L or more of fluid, which represents a significant weight that can compress, squish, or deform the patient's soft tissue. Therefore, it may be desirable to perform pre-operative imaging with such an acoustic coupling container in place, such that any deformations that will be seen during the procedure will also show up on pre-operative imaging. In some embodiments, however, this may not be practical or possible. Therefore, in some embodiments, treatment planning can be based on pre-operative imaging, and once the patient is on the operating table with the acoustic coupling container filled and in place, additional imaging can be taken to identify the deformations from the acoustic coupling container. The system can then be configured to co-register the pre-operative images (upon which the treatment planning is based) with the images that are deformed by compression of the acoustic coupling container, and make any adjustments to the treatment plan, target tissue volume, and/or pose or angle of the treatment head based on these deformations and the co-registration.

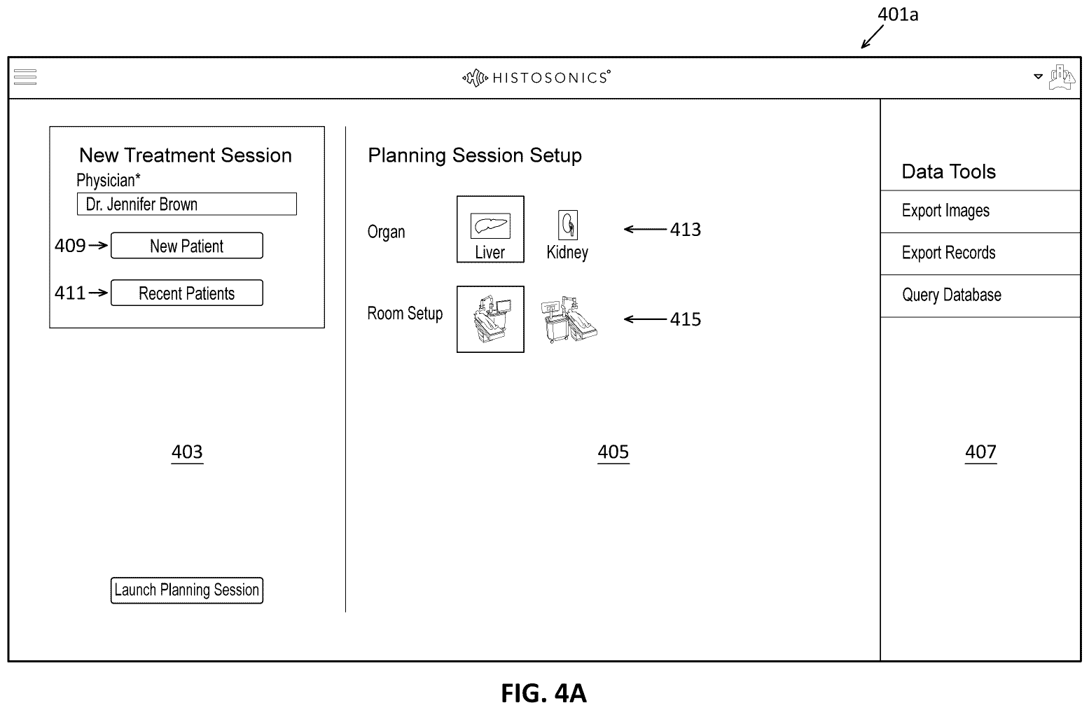

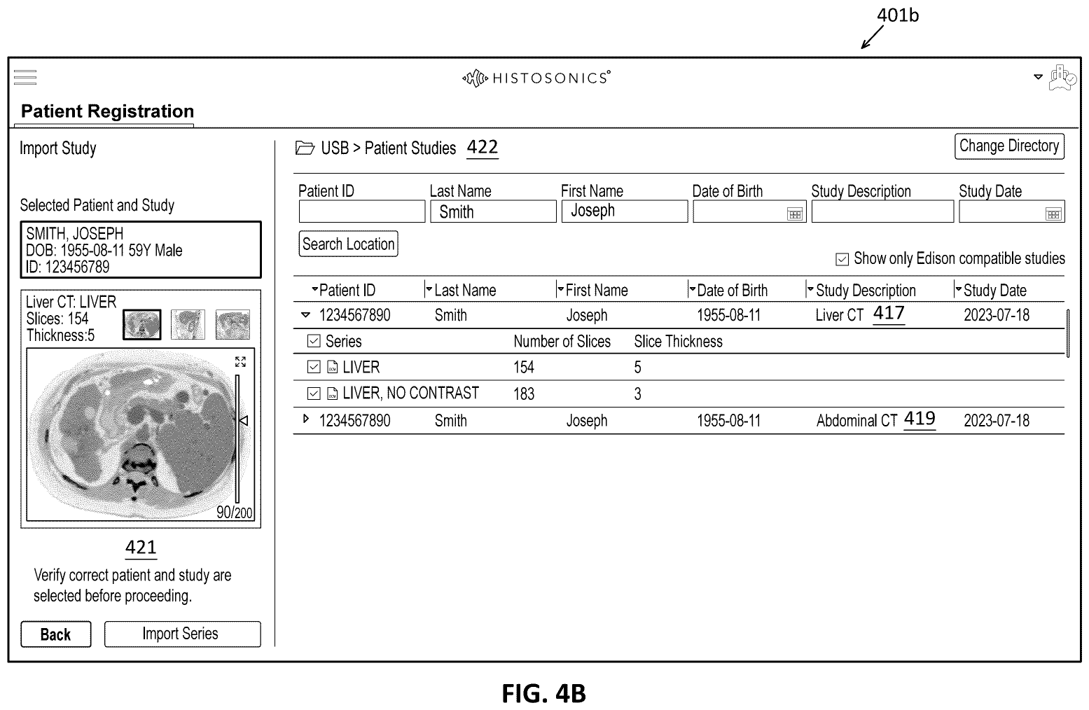

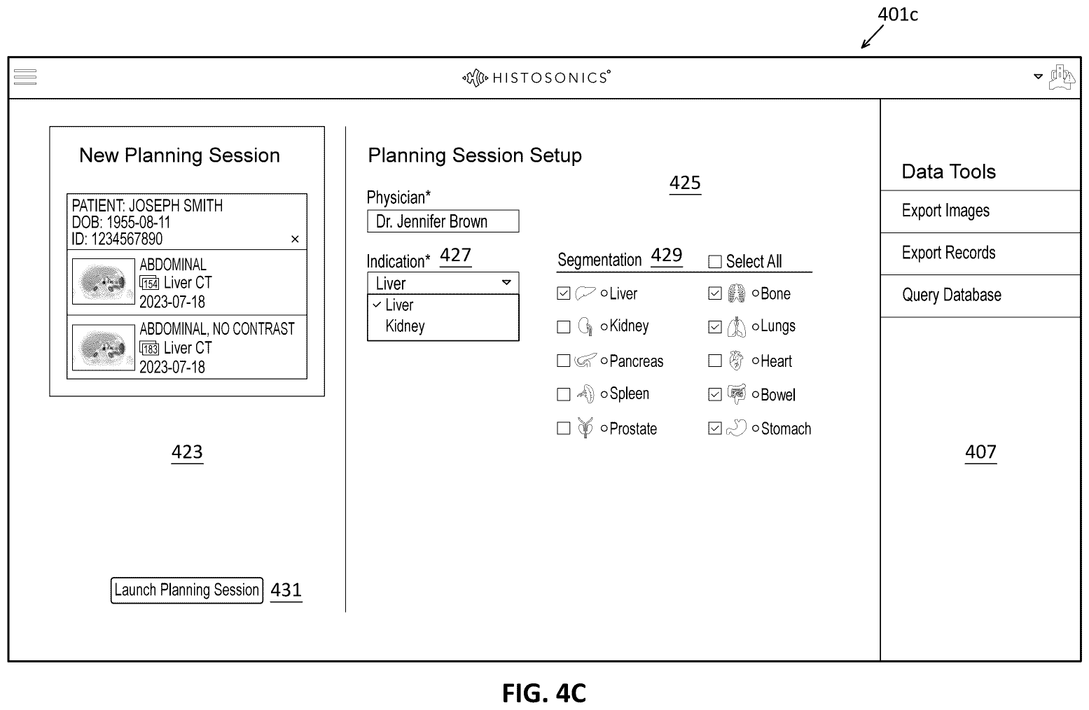

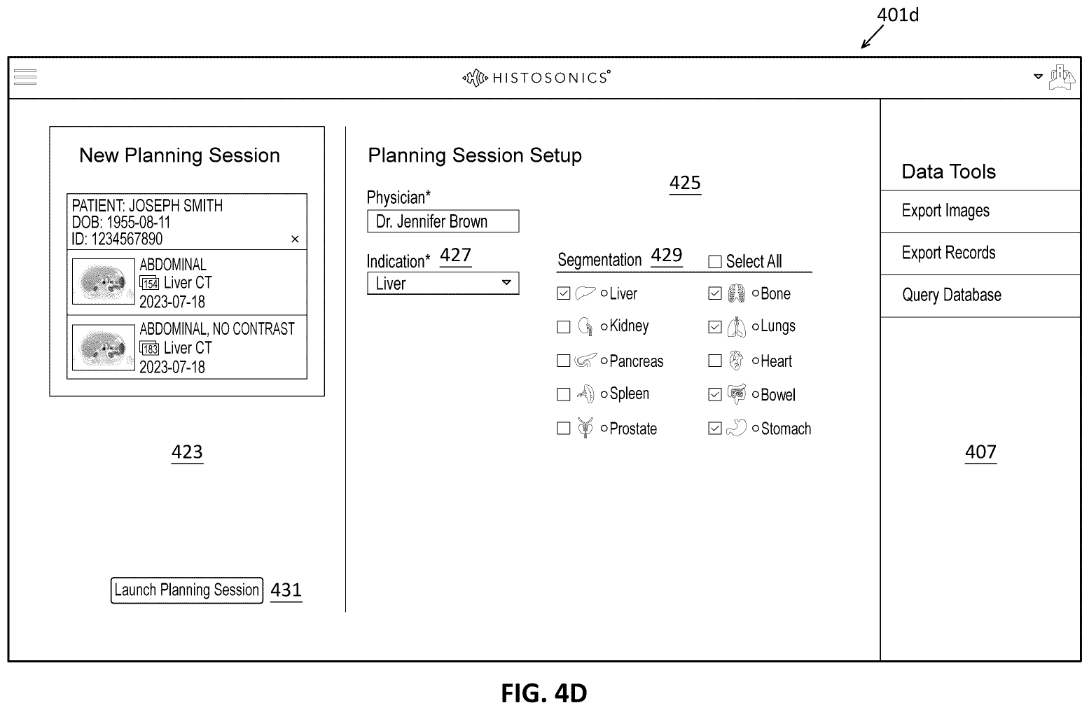

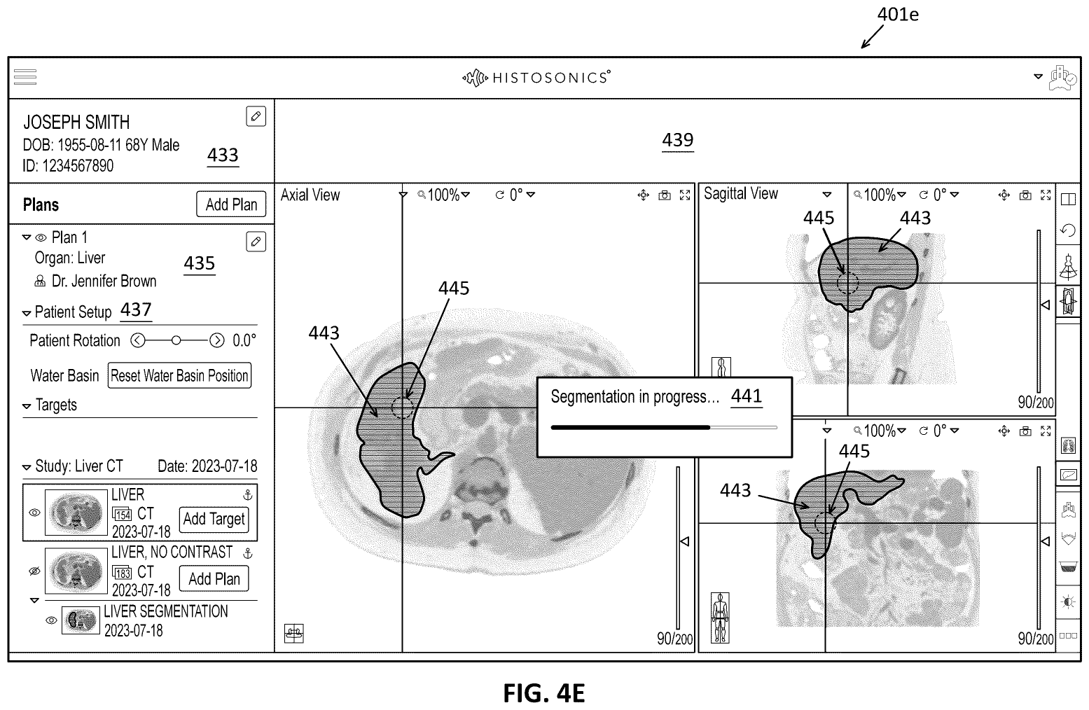

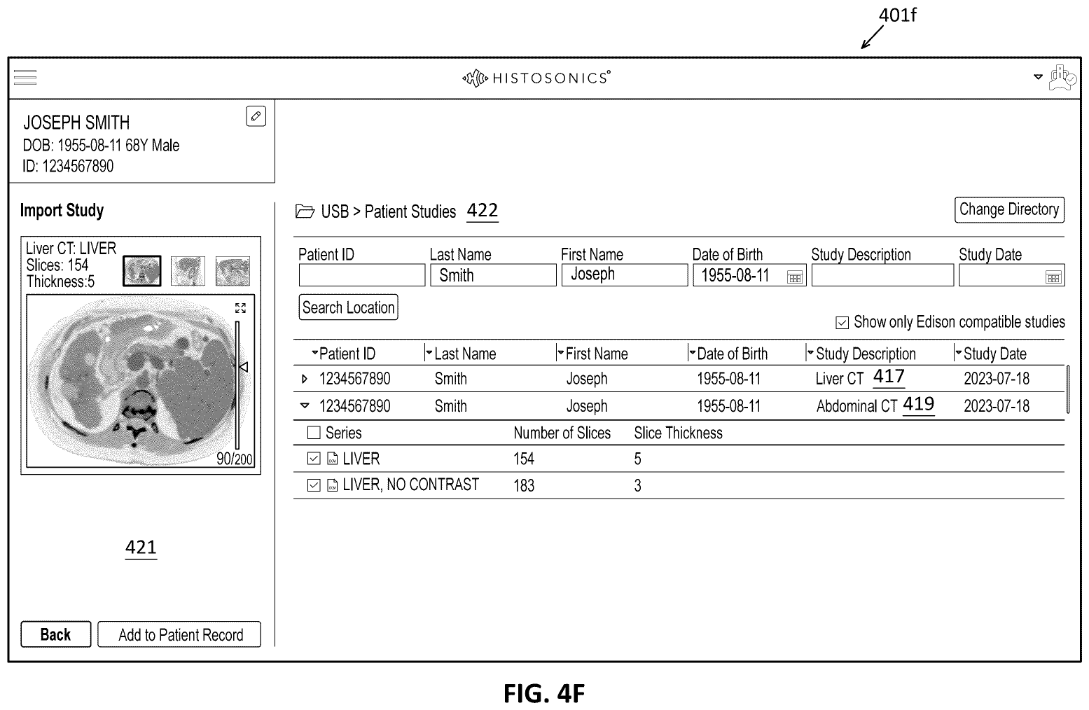

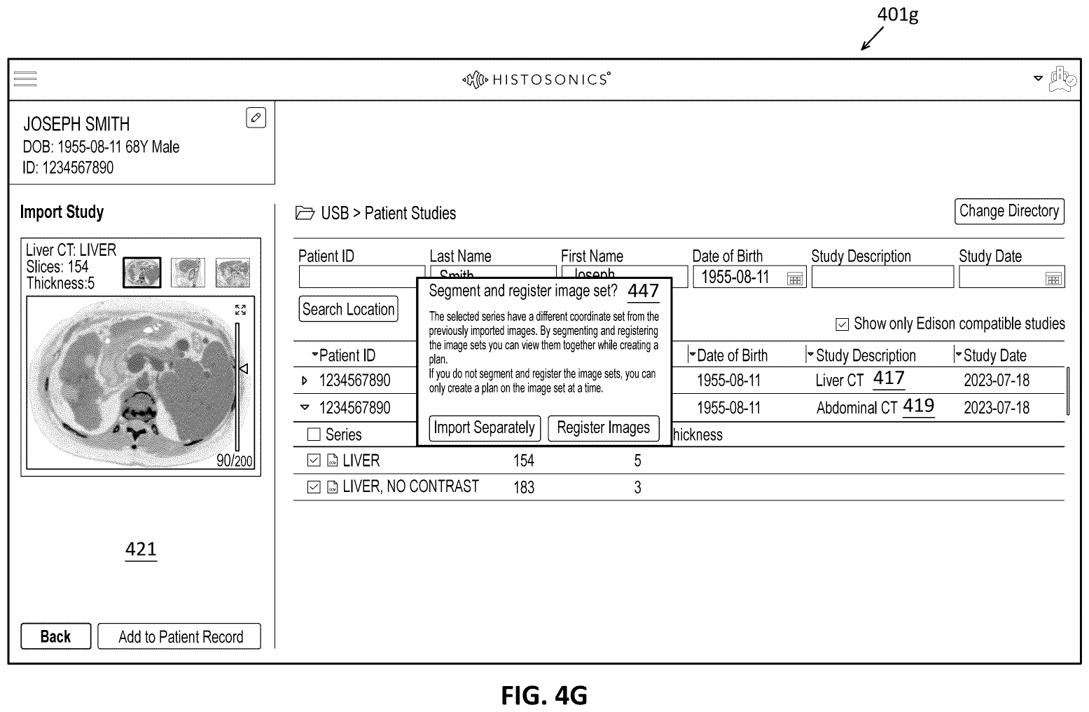

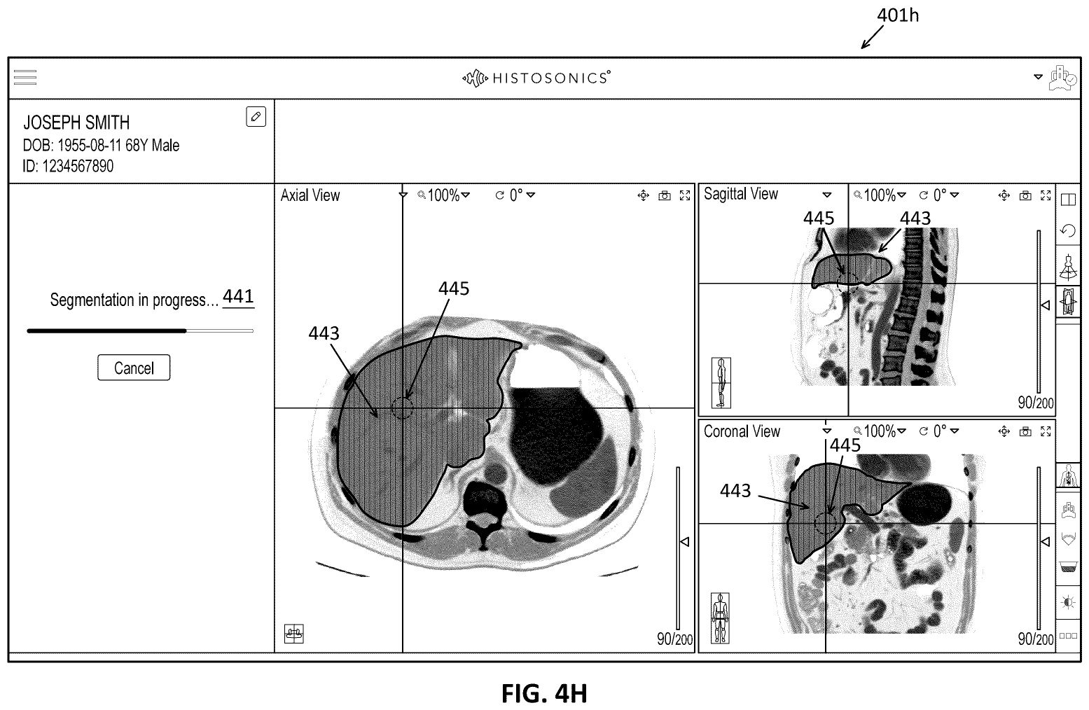

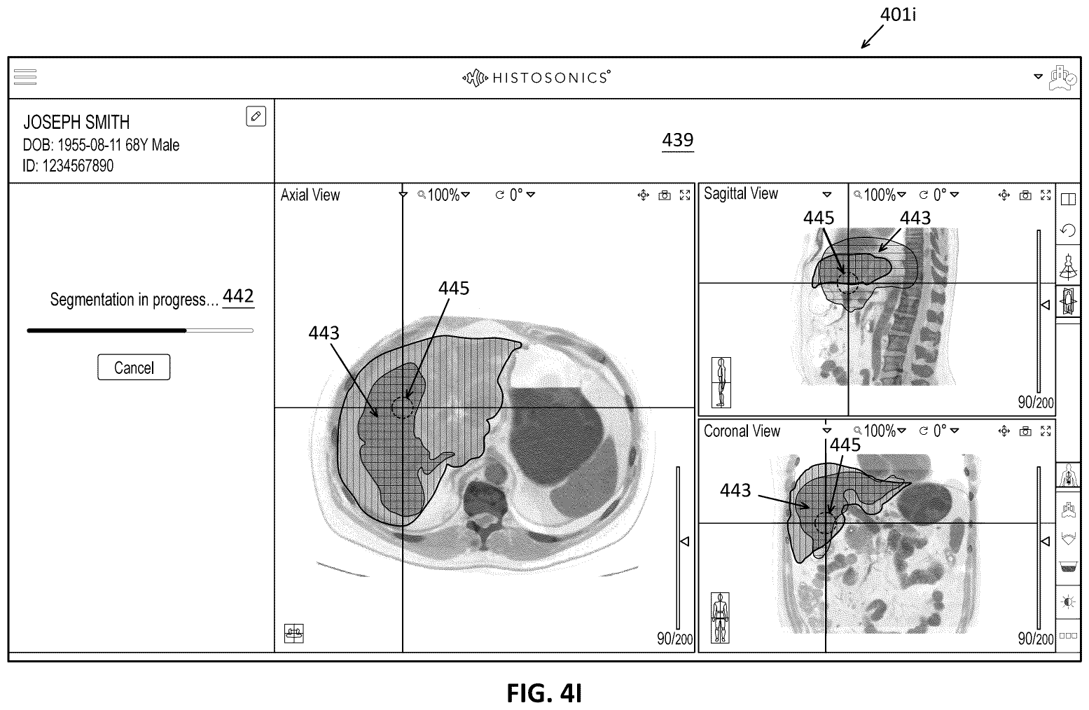

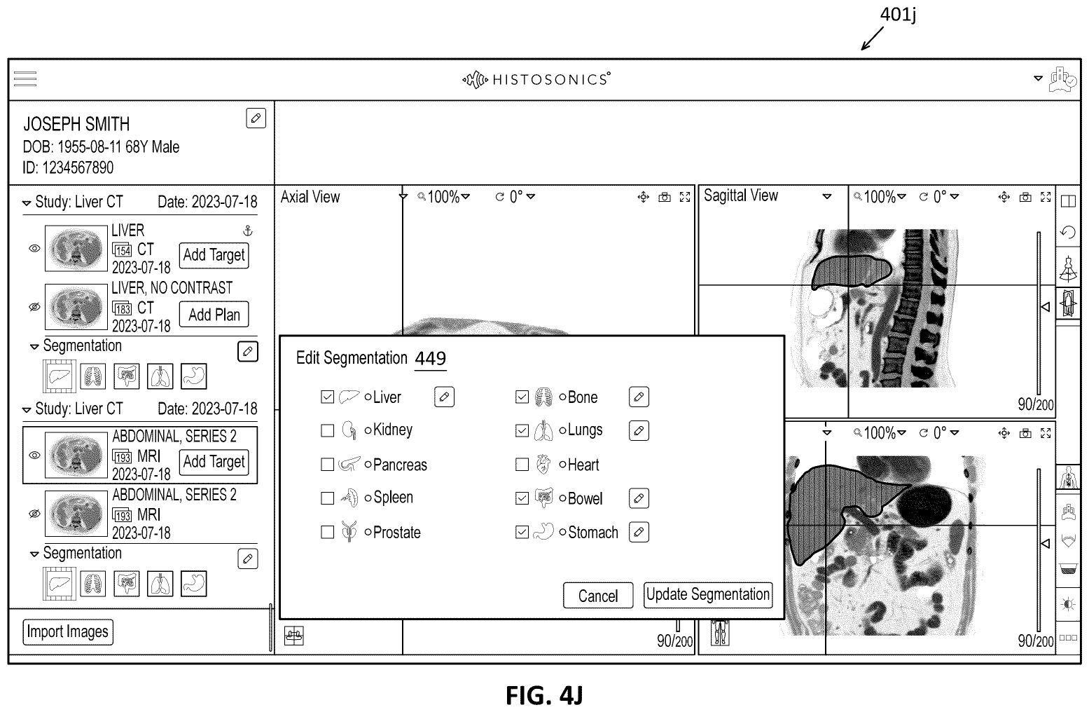

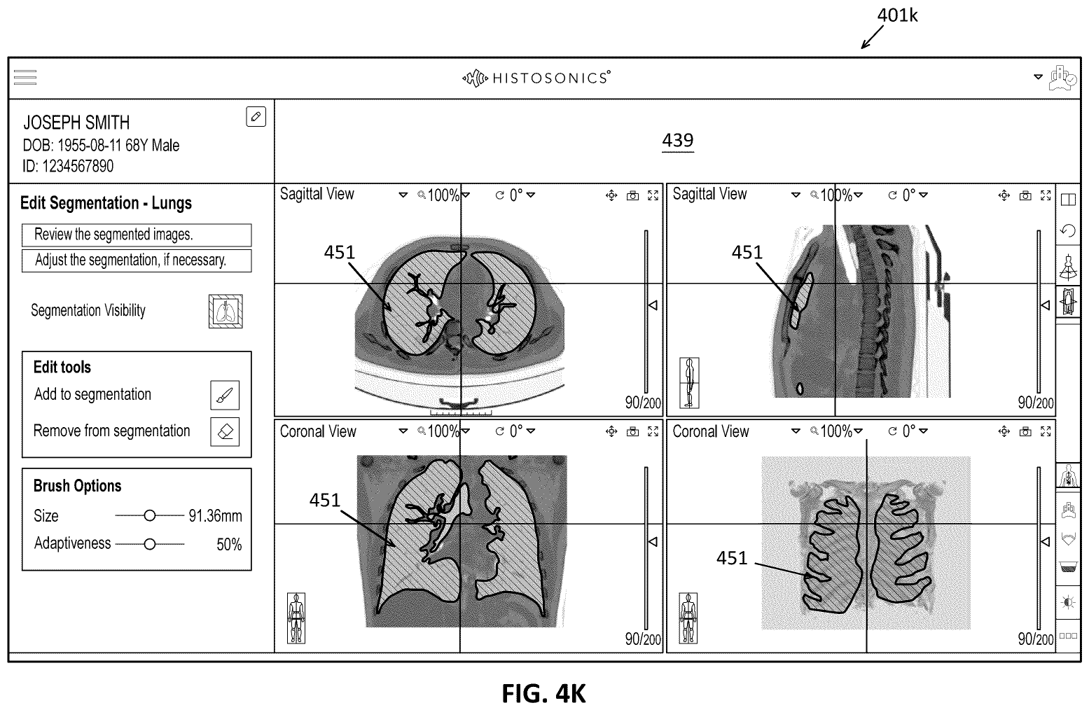

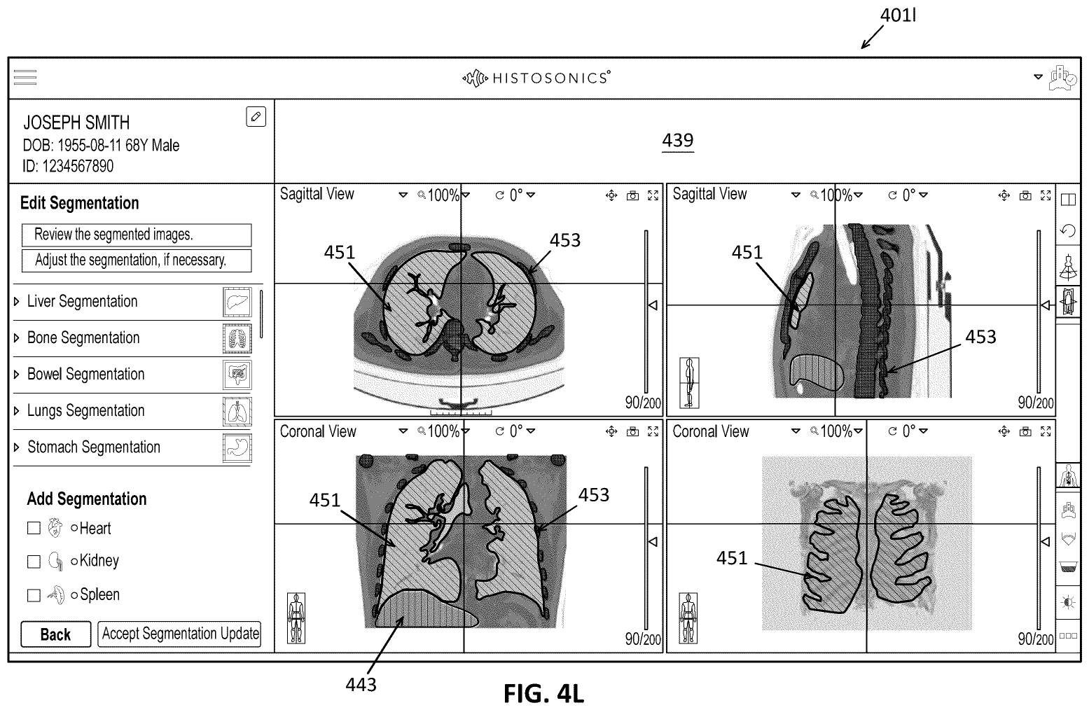

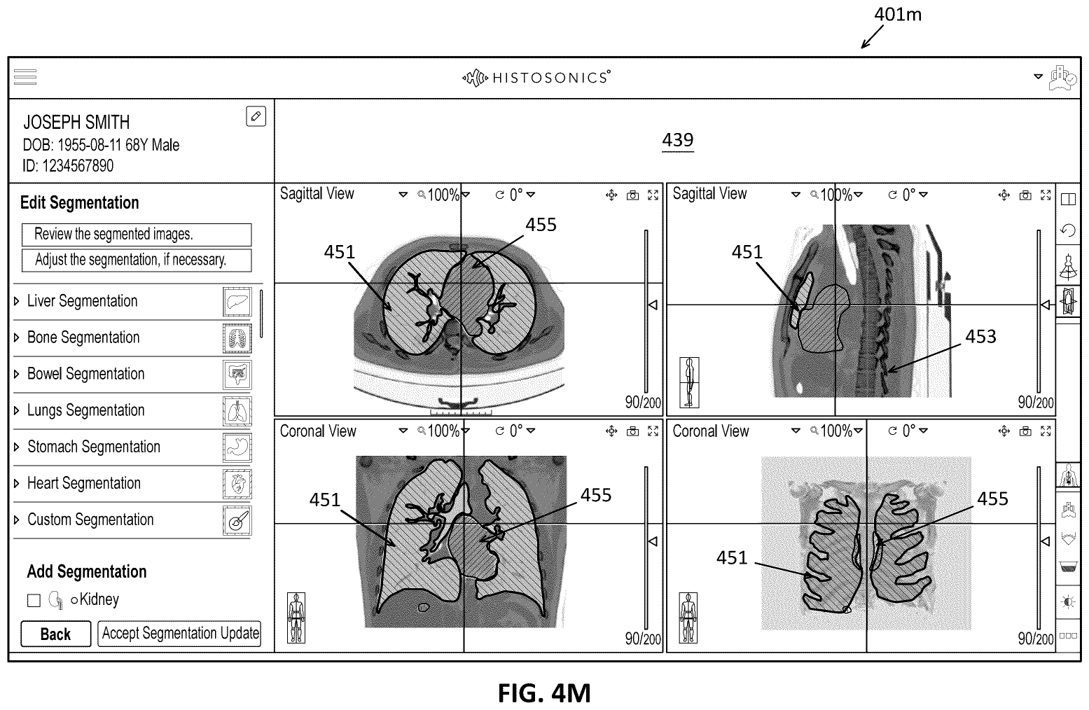

Referring to FIG. 4A, a user-interface (UI) screen 401a of a treatment planning user interface is provided. As described above, treatment planning tools of the present disclosure can be implemented in software on a console of the histotripsy system or in a remote computing environment such as the cloud or a personal device. The screen 401a can be displayed to a user of the treatment planning tool in a display such as a monitor or television. In some embodiments, the screen can be implemented as a graphical user interface (GUI) which allows the user to directly interact with the screen (e.g., via a touch screen). In other examples, the user can interact with the screen with peripheral input devices, including but not limited to joysticks, computer mice, keyboards, touch pads, buttons, dials, knobs, or custom input devices.

In the illustrated embodiment, the UI screen 401a is divided into a plurality of sections, including New Treatment Session section 403, Planning Session Setup section 405, and Data Tools section 407. In the New Treatment Session section 403, a user, such as the treating or planning physician, can enter their credentials and identify if the patient to be treated is a new patient 409 or a recent or previously treated patient 411.

In the Planning Session Setup section 405, the user can identify the organ to be treated at location 413. In the illustrated example, the user has a choice between liver treatment or kidney treatment, but it should be understood that any number of organs or treatment locations can be selected in this section of the UI screen, including pancreas, spleen, prostate, bone, lungs, heart, bowel, stomach, brain, etc. The organ/treatment location to be treated can in some embodiments customize the UI screen and workflow steps that follow to be specifically tailored to the selected organ/location. For example, a treatment workflow for treating the liver or a liver tumor may be different than a treatment workflow for treating the kidney or a kidney tumor.

Also in the Planning Session Setup section 405, the user can identify or choose the treatment room setup at location 415. In the illustrated example, the location 415 provides a right-hand setup option (with the histotripsy console positioned to the right of the treatment table) and a left-hand setup option (with the histotripsy console positioned to the left of the treatment table). Other treatment room setups are contemplated and can be provided in this section, including treatment room setups that included additional hardware or functionality including but not limited to CBCT imaging, X-ray imaging, separate fluidics management systems, or other surgical systems or robots such as, for example, Da Vinci robotic surgical systems by Intuitive Surgical or the like.

Data Tools section 407 provides the user with access to medical imaging and records, including previously obtained medical imaging such as CBCT, CT, MRI, X-ray, or ultrasound imaging. The Data Tools section 407 allows the user to import or export these images and access additional data or records as-needed. The data accessible via Data Tools section 407 can be stored locally, on the cloud, or on other devices such as portable hard drives.

Once a new planning session has been started with a selected patient, FIG. 4B shows a UI screen 401b that presents the user of the planning tool with a listing of imaging sets available for the treatment planning process in Patient Studies section 422. In this example, there are two separate imaging sets available for the selected patient, a Liver CT scan 417 and an Abdominal CT scan 419. Section 421 in this example can provide a preview of the imaging, which in this case is the selected Liver CT scan 419. The user is prompted to confirm the proper imaging selection before proceeding in the treatment planning workflow.

In FIGS. 4C-4D, UI screens 401c/d respectively are shown which, in New Planning Section 423, provides patient information and the selected imaging sets, and in Planning Session Setup section 425, provides the user with selectable options for the treatment planning, including selecting the indication 427 from a drop-down menu and also selecting the tissue types or organs that are to be segmented by the treatment planning software in Segmentation section 429. The drop-down menu for indication 427 can list all the same choices as in location 413 of Planning Session Setup section 405 discussed above in FIG. 4A. In this example, kidney and liver are selectable options, but as mentioned above other indications or treatment locations/organs are contemplated.

Segmentation section 429 provides a non-exhaustive list of organs to be segmented in the imaging sets, including in this example liver, kidney, pancreas, spleen, prostate, bone, lungs, heart, bowel, and stomach. The segmentation process (described below) can apply color coded or other visually distinguished segmentation of selected tissues/organs to the user for easy identification on the selected medical imaging. Once the indication 427 and segmentation section 429 options have been selected by the user, interacting with Launch Planning Session icon 431 can proceed the treatment planning tool to the next step of the process.

FIG. 4E shows a UI screen 401e once the user has launched the planning session with the selected indication and segmentation options. In this example, the UI screen 401e includes a Patient Information section 433 which provides relevant information including patient name, age, date of birth, gender, and patient ID. Plan section 435 provides information on the current treatment plan, target organ, and operating physician. Patient setup section 437 provides information on the patient setup and arrangement on the operating table, such as the angle of rotation on the operating table and the size and/or filling volume of the acoustic coupling container. In the illustrated example, the planning is based on the patient being not rotated on the operating table (e.g., the patient laying flat on his or her back). If the target tissue location requires a different orientation, it can be reflected by adjusting the slider or rotation value in section 437.

Image segmentation section 439 of UI screen 401e shows the chosen indication or organ of interest 443 (e.g., liver) being segmented 441 in the imaging set (e.g., the liver CT 417 from FIG. 4B) in one or more views (e.g., axial, sagittal, coronal). In some embodiments, the segmentation can include enhancing or adding a visual indicator to the segmented organ, such color coding, outlining, increasing the contrast, or other techniques for presenting a segmented portion of an image to a user. For example, in one example, the segmented organ can be highlighted in a distinct color such as blue, orange, red, etc. In other embodiments, the segmented organ can be outlined, such as with solid or dashed lines.

Additionally, as shown in image segmentation section 439 of UI 401e, a target tumor or target tissue volume 445 within the chosen organ can be identified and/or segmented. In some examples, the system can apply additional coloring, outlines, or contrast adjustments to the tumor or target tissue volume. Additionally, as shown, the system can apply cross-hairs or lines in the horizontal and/or vertical directions to pinpoint the location of the tumor or target tissue volume within the segmented organ.

FIG. 4F shows an additional UI screen 401f in situations where the user of the treatment planning tool wants to incorporate additional imaging sets and/or segmentations into the treatment planning workflow. In this example, the UI screen 401f has returned to the menu that shows Patient Studies section 422 (described above in FIG. 4B). In the prior UI screens, the user had chosen the Liver CT scan 417 for importation and segmentation by the treatment planning tool. Now, in FIG. 4F, the user can select the other imaging set, Abdominal CT scan 419, for use by the planning tool.

Upon selecting another imaging set, at UI screen 401g of FIG. 4G, the system and UI screen can indicate 447 to the user that the selected imaging set has a different coordinate frame of reference from the previously imported images. This can be a result of, for example, the various imaging sets being obtained along different sections of the sagittal, coronal, or axial planes of the patient. For example, the Liver CT scan 417 may be taken in an axial plane superior to (e.g., +) from the axial plane in which the Abdominal CT scan 419 is taken, or vis versa. Since the imaging sets may be in different coordinate frames of reference, co-registration of the imaging sets to share a common coordinate frame of reference is required for treatment planning that uses more than one coordinate frame of reference. If the user chooses not to co-register the imaging sets, then the treatment planning can only proceed with a single imaging set.

FIG. 4H shows a UI screen 401h segmenting 441 the chosen indication 443 (e.g., liver) in the second imaging set. As described above, the segmented organ can be colored, highlighted, shown with increased contrast, or outlined. Additionally, the target tissue volume or tumor 445 can also be colored, highlighted, outlined, or shown with increased contrast, and further identified with cross-hairs or horizontal and/or vertical lines on the images.

FIG. 4I shows a UI screen 401i with the co-registration of the first imaging set (Liver CT scan 417) being registered with the second imaging set (Abdominal CT scan 419). The indication 443 and target tissue volume 445 can also be shown in this co-registered image, as described above.

Referring to FIG. 4J, in UI screen 401j, the user has the option to re-segment any chosen indication, even after the segmentation of one or more targets or organs has been performed. For example, Edit Segmentation menu 449 may allow the user to segment any of the organs or targets previously described, including but not limited to liver, kidney, pancreas, spleen, prostate, bone, lungs, heart, bowel, or stomach. Section 442 shows that a Segmentation of selected organs or tissue types is in process.

FIG. 4K shows a UI screen 401k in which the indication 451 of the lungs are segmented in the image(s), FIG. 4L shows a UI screen 401l in which the indication 443 of the liver, indication 451 of the lungs, and indication 453 of the bones are segmented in the image(s), and FIG. 4M shows a UI screen 401m in which the indication 451 of the lungs, indication 455 of the heart, and a custom segmentation 457 are segmented and shown in the image(s). In this example, the custom segmentation 457 can be an annotation, marking, or manual segmentation performed by the user of the system. For example, the user may circle or highlight the target tissue volume or tumor from the prior liver images in this image set, to view the location of the tumor relative to other important structures or organs such as the lungs and heart.

Any of the treatment planning tools described herein can be used to generate and display a 3D digital model of the patient, and can include, overlay, or co-register any medical imaging of the patient with the 3D model (CT, MRI, PET-CT, fusions of any imaging, etc.). The 3D model can include segmented target organs or tissues, as selected by the user, or automatically selected by the software workflow for a given target treatment location. Segmentation of any user-selected organ or tissue allows for the user/physician to not only assess the target tissue location, but also assess adjacent tissue/organ structures.

### Simulation of Operative Tools

The treatment planning tools provided herein are configured to simulate the position and orientation of operative tools and systems relative to the patient, such as the therapy transducer and/or robotic arm, acoustic coupling container, etc. The planning tools allow a user or the system to adjust or modify the position, pose, and/or orientation of the therapy transducer relative to the patient and/or to the acoustic coupling container to identify optimal or preferred acoustic windows for the therapy transducer to a target treatment location. The planning tools can use ray tracing or other simulation techniques to identify potential obstructions within the patient along the therapy transducer acoustic path. A user or the system may use the planning tools to adjust the positions of the therapy transducer, robotic arm, and acoustic coupling container and identify any potential collisions between the transducer array, the robotic positioning arm, the patient, and any other external structures such as the acoustic coupling containers or other patient support structures including the operating room table.

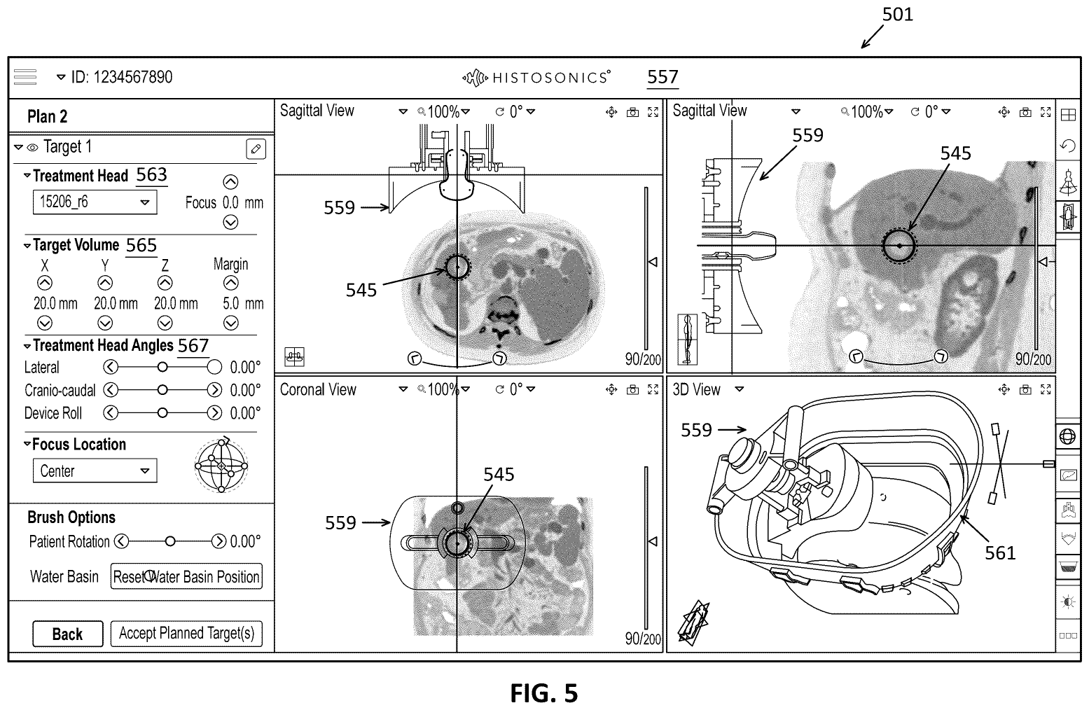

Referring now to FIG. 5, a UI screen 501 is shown which can provide a graphical representation of one or more operative tools or structures relative to the patient imaging including the target tissue volume or tumor 545 (previously discussed as target tissue volume 445 above). In FIG. 5, image planning section 557 can provide a variety of views of the segmented and co-registered imaging sets described above, along with graphical representations of a therapy transducer array 559 and/or additional surgical equipment or components including, for example, acoustic coupling container 561. In the illustrated embodiment, image planning section 557 provides axial, sagittal, coronal, and 3D views, which can be user-selectable depending on the user's preference.

UI screen 501 can include a Treatment Head section 563 which may allow the user to select a desired treatment head for the target tissue volume 545, such as from a drop-down menu. Different histotripsy treatment heads may be available for use with the histotripsy system depending on the size, depth, or tissue type of the target tissue volume. For example, smaller treatment heads may be desirable for smaller or shallower targets, while larger treatment heads may be required for deeper tissue volumes, or volumes that are hidden behind or obstructed by bone or other obstructions within the tissue. The larger treatment heads may be capable of higher energy delivery levels which can account for aberrations or obstructions between the transducer and the target tissue volume. In some embodiments, the treatment head can be automatically chosen by the system based on the depth of the target tissue volume 545. For example, when the user selects the target tissue volume 545, the treatment planning tool can calculate the depth of the targe tissue volume, and automatically select the treatment head with the appropriate focal length for that depth. The type of acoustic coupling system/container can factor into the selection of the treatment head. For example, certain anatomies may facilitate coupling systems that allow the treatment head to be placed closer to the patient's skin, while other anatomies may require the treatment head to be further away. The type of coupling system used can factor into the depth between the treatment head and the target tissue, and therefore into the type of treatment head that is appropriate for that treatment location.

Target Volume section 565 may allow a user to adjust the size of the target tissue volume 545, including any additional margins to include in the treatment plan. The size of the volume can be adjusted in X, Y, and Z dimensions, at a pre-selected interval (e.g., in increments of 0.1 mm or less).

Treatment Head Angles section 567 allows the user to manipulate the position and/or orientation of the virtual treatment head, providing adjustments along any number of pose/angle parameters such as lateral, cranio-caudal, and device roll adjustments. In some examples, adjustment of these parameters also causes the graphical representation of the therapy transducer array 559 to move in real-time on UI screen 501. In some embodiments, the position and/or orientation of the virtual treatment head can represent a starting point for the planned histotripsy therapy. The coordinates of the pose/position of the simulated or virtual treatment head can be saved into the treatment plan. When the treatment plan is loaded into the histotripsy system for actual treatment, the robotic arm and/or treatment head of the actual histotripsy system can automatically position itself at the pose/position coordinates chosen by the user during the treatment planning process.

In any of the embodiments described herein, the transducer array pose/angle and/or position, along with the target volume size and margins can be saved into a given treatment plan and transferred to a histotripsy system for histotripsy therapy. When the treatment plan is loaded into the histotripsy system, all the selections made by the user, including transducer array pose/position, tissue volume size and margins, treatment head type, and any of the other parameters described above are automatically loaded into the histotripsy console to enable the system to provide therapy according to the treatment plan.

### Obstruction Detection

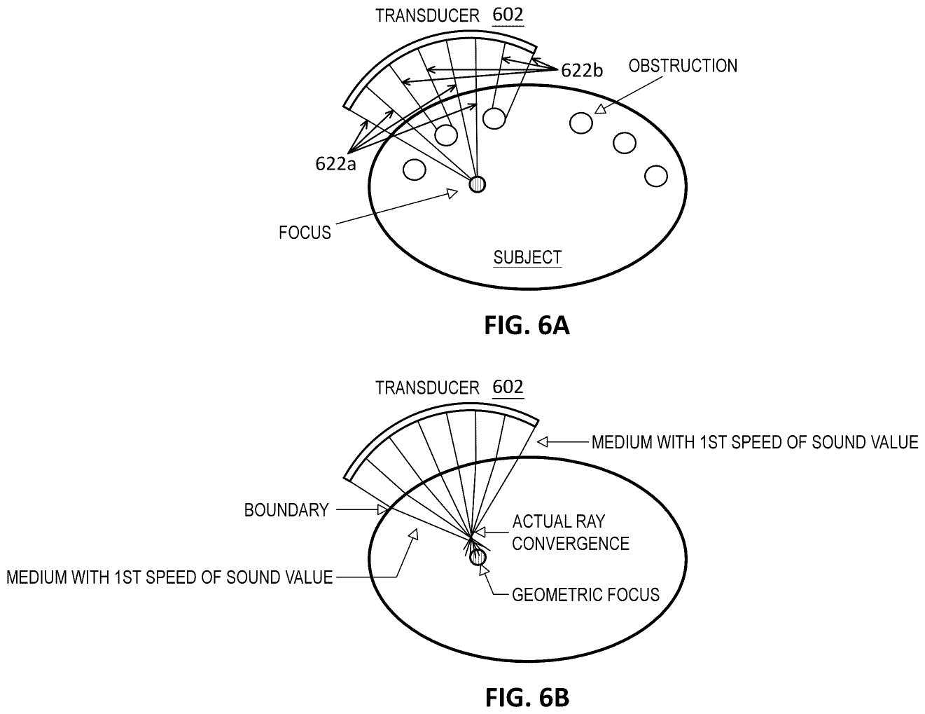

As described above, bone and gas in the beam path of the transducer can block or divert transmission of acoustic energy from the target focal location, reducing the total acoustic pressure developed. This can potentially reduce the developed acoustic pressure to below the threshold for therapeutic effectiveness or cavitation generation. Such obstructions may also pose a risk for thermal injury, either by creating hot spots in intervening tissue and/or requiring the deposit of more acoustic energy into the tissue. FIG. 6A is an illustration of an ultrasound transducer array 602 positioned adjacent to the skin of a subject. Individual transducer elements of the transducer array 602 can be directed towards a focus within the subject, as shown with individual rays being emitted from the array. One or more obstructions between the transducer and the focus are illustrated, such as bone (ribs) and/or gas within the body. These obstructions can block the propagation of acoustic energy to the focus. For example, in FIG. 6A, transducer emissions 622a are unobstructed and reach the focus while transducer emissions 622b are blocked by one or more obstructions within the subject.

Changes in speed of sound along the propagation path of the transducer array emissions can also affect where energy is deposited. For example, referring to FIG. 6B, as emissions pass through a boundary where the speed of sound changes, then the angle or direction of the emissions can change due to refraction. When ultrasound waves move between two different mediums where the speed of sound increases, such as the boundary between water (transducer coupling) and soft tissue, then the emissions will tend to converge at point closer to the transducer than its geometric focus. This will affect where subsequent boundary interceptions will occur and also the location of any focal therapeutic effects. It is important to know where the actual therapeutic focus is so that energy and cavitation can be directed to targeted tissues to ensure that a target tissue volume is fully treated.

To address these issues, this disclosure provides intuitive and interactive visual and quantitative feedback in the form of user-facing treatment planning tools to identify an optimal or preferred acoustic window, transducer position and orientation (pose), including systems and methods for visualizing the shadows of obstructions as projections on the skin and/or transducer surface.

### Ray Tracing

Ray tracing can be used in some implementations of a treatment planning tool to simulate the propagation of acoustic energy from the histotripsy transducer array through patient anatomy. As described above, the treatment planning tool can use high resolution medical imaging (e.g., CT, CBCT, MRI, X-ray, etc.) and perform the ray tracing on the medical imaging from a simulated therapy transducer array towards a focus or target tissue volume within the patient. The medical imaging can be performed on the patient prior to or during the procedure.

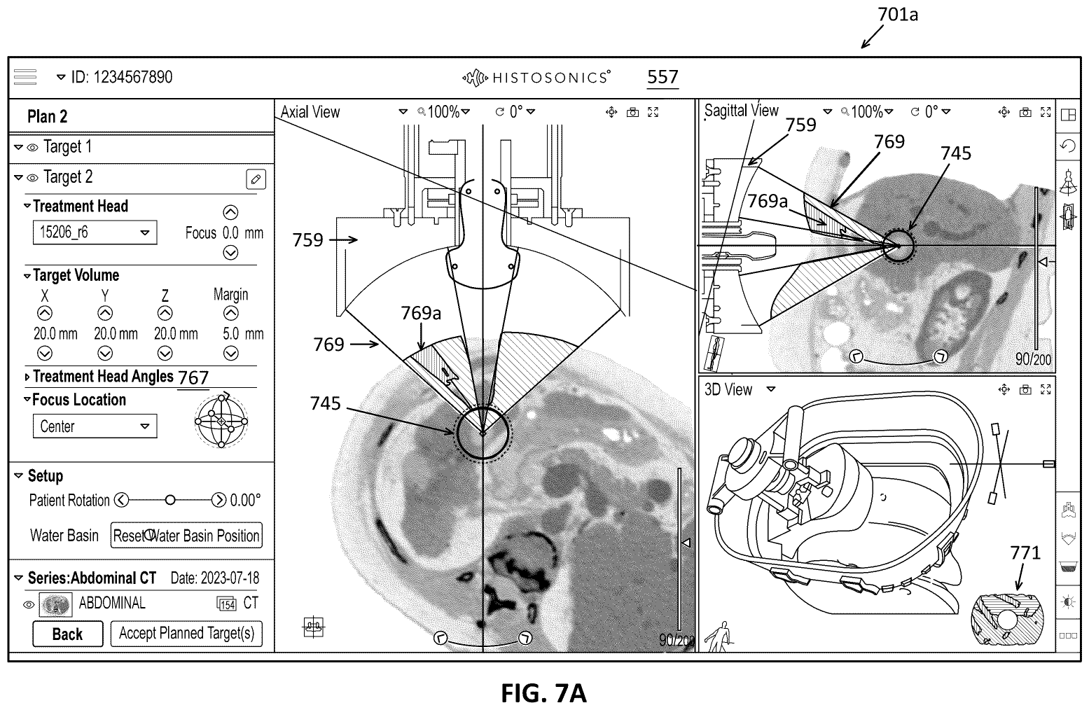

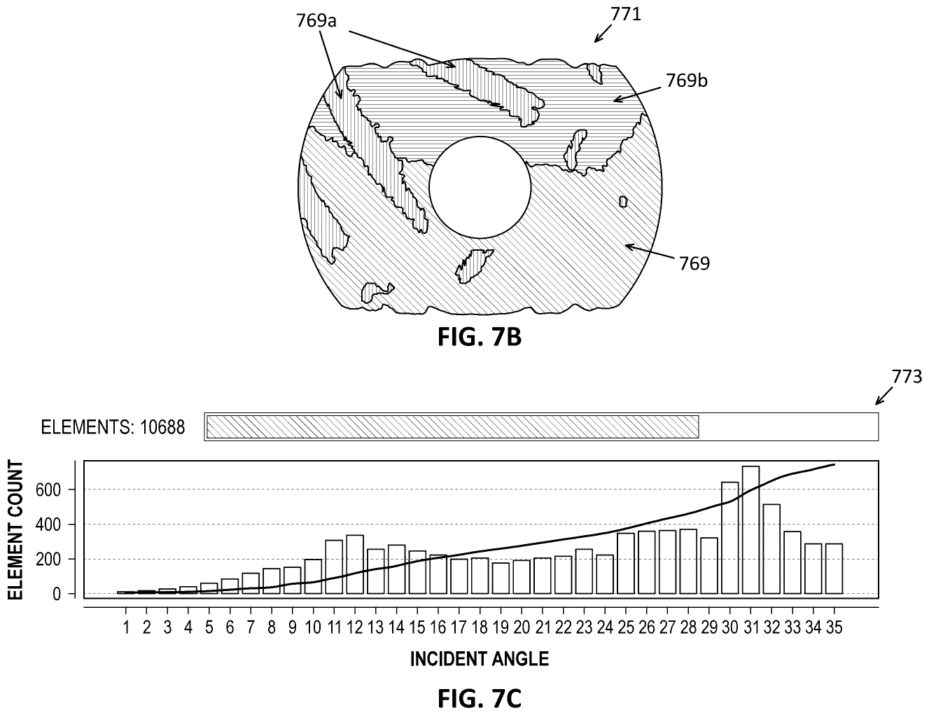

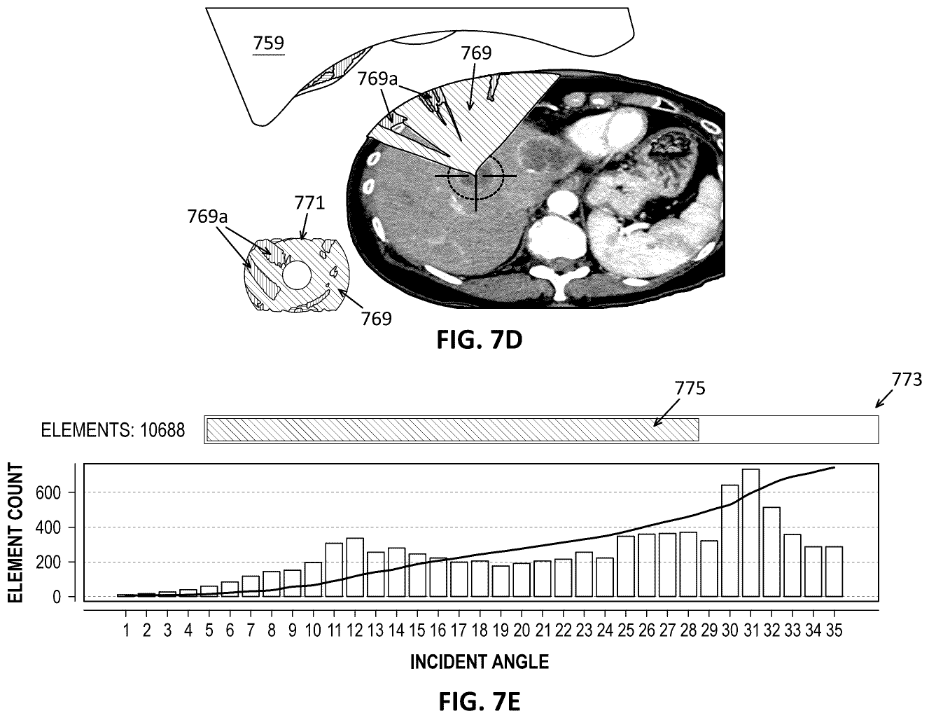

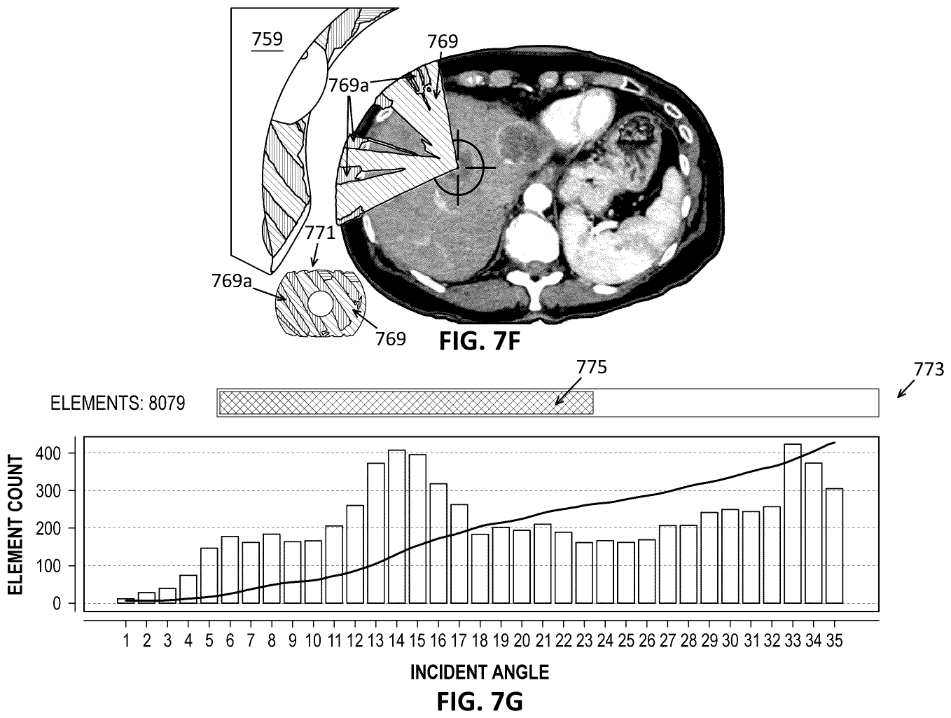

In one example, referring to FIG. 7A, the emitting surface of a simulated transducer array 759 representing a histotripsy therapy array can be finely sampled by a set of evenly spaced points, each a starting point for a normally oriented ray 769. The points can correspond, for example, to individual transducer elements of the transducer array. Each ray may be traced through medical imaging (e.g., CT imaging) of the patient or subject until it reaches closest approach to the geometric focus within or at the target tissue volume 745, or otherwise terminates (e.g., at an obstruction). As each ray is stepped along its path, the patient image data can be sampled from pre-treatment images (e.g., CT, MRI, etc.) in order to determine if any anatomical structure boundaries are detected. These structures can be segmented prior to ray tracing (as described above) or in the case of CT can be identified by Hounsfield value thresholds.

In one implementation, referring to FIG. 7A, the transducer can be sampled across its face to provide starting points for tracing rays 769. Patient specific volumetric medical imaging can be used to identify some or all of tissue types, tissue densities, and/or obstructions (e.g., bone, pockets of gas or air, medical implants, etc.). The medical imaging can comprise, for example, MRI or CT images of the subject as discussed above. The rays can be oriented normal to the transducer surface and can be traced through the patient specific volumetric images, typically CT or MR, until tissue boundaries and/or obstructions are encountered. Rays that pass-through obstructions such as bone or gas (e.g., rays 769a) can be marked as obstructed with a visual indicator, such as a colormetric indicator or any other visual indicator such as increased or reduced contrast, highlighting, outlining, or the like.

As described above, refraction of the rays can be applied using Snell's law in the cases where a ray moves across a structure boundary where the speed of sound changes. In FIG. 7A, ray tracing can be used to identify rays which cross a boundary where the speed of sound changes and are refracted according to Snell's law. This generally means that the rays do not terminate at the geometric focus and terminate instead at their closest approach to the focus. In the case where the speed of sound increases as the rays cross a boundary (e.g. water to soft tissue), the rays are refracted such that they converge at a point closer to the transducer than the geometric focus. For non-invasive therapies, the histotripsy transducer array is typically positioned outside the subject and acoustically coupled to the patient with a coupling medium such as water, saline, acoustic coupling gel, or the like. The speed of sound properties of the acoustic coupling medium are known, and the speed of sound properties of tissue are generally known or can be approximated. Therefore, the interface or boundary between the coupling medium and the subject's skin represents a potential boundary where refraction of rays can occur.

The results of the ray tracing procedures described above can be visualized in a treatment planning tool associated with a histotripsy system in several useful ways. In one example, GPU implementation of ray tracing enables interactive updates as the transducer or patient is moved around making for a fluid user experience. The ray tracing can be visualized purely in a treatment planning environment, e.g., by moving a simulated transducer around patient imaging, or alternatively, can be implemented in real-time on a graphical user interface of a histotripsy system while the patient is present and undergoing or about to undergo histotripsy therapy.

In the example of FIG. 7A, the system can display the ray paths for a given histotripsy transducer array (simulated or real) in relation to the transducer and patient anatomy. Ray color or other visual features, colors or patterns can be used to indicate various properties like obstruction or incidence angle. For example, UI screen 701a of FIG. 7A shows rays 769 from a simulated transducer array 759 displayed in a first color such as green can indicate to a user that a given transducer element of the array is unobstructed and arriving at the target location. The rays can be presented as being emitted from the transducer array 759 and into the medical or patient imaging of the patient terminating at the focus or the target tissue volume 745, as shown. Rays 769a displayed in another color, such as red, can indicate that a transducer clement is blocked or obstructed. Other colors can be used to convey information to a user. For example, a yellow ray may indicate a transducer element that is partially blocked, or alternatively, a ray that is refracted due to speed of sound changes and therefore may not pass through the geometric focus of the transducer array. A blue ray may be used to indicate an air pocket or air-filled organ such as the lungs. While the embodiments discussed above describe using colors to convey the various tissue types and/or obstructions, it should be understood that other visual techniques can be used to convey the same information. For example, the rays could be greyscale with the darkness (or lightness) of the rays being used to convey how much of the ray reaches the focus of the transducer array. Alternatively, dashes, dots, or other patterns in the rays can be used to convey the same information. In some examples, hybrid visualizations can present the ray paths in combination with volume rendering of obstructing structures like the rib.

The user interface can include additional visualization tools for a user during treatment planning to assist in decision making on transducer pose angle and positioning. In one example, a simple 2D view 771 of the ray tracing data may be mapped back to a 2D view of the transducer array face to provide a useful heads-up display on the UI screen 701a. FIG. 7B is a close-up view of the 2D view 771, which provides the rays mapped to a 2D view of the transducer array face and includes information on the ray tracing analysis with color coded rays 769, 769a, and 769b representing unobstructed rays, obstructed rays, and rays passing through pockets of gas or other partial obstructions, respectively.

In some embodiments, the UI screen 701a can further provide a simple graph or meter 773, shown in FIG. 7C, which indicates the relative amount of transducer elements that are unobstructed or obstructed. This graph or meter 773 can be implemented in a number of ways, including using color coding (e.g., green=most elements unobstructed, yellow=some elements obstructed, red=many elements obstructed) as a percentage or ratio of unobstructed to obstructed elements, or simply a count or quantification of the amount of unobstructed transducer elements (similar to a fuel gauge). This can provide the user with a real-time indication as to which portions of the transducer array are blocked, partially blocked, or unobstructed. Additionally, the user can get instant feedback on the amount or proportion of transducer elements that are unobstructed/obstructed.

In the simulated environment described herein, the user can manipulate the position and orientation of the simulated transducer array 759 within Treatment Head Angles section 767 to try to find a more optimal acoustic window. For example, FIG. 7D shows a first pose of the transducer array 759 with 2D view 771 showing mostly “green” or unobstructed rays traced back to the transducer surface with several strips or patches of “red” corresponding to, for example, ribs blocking transducer elements in the current pose. In FIG. 7E, the graph or meter 773 shows that most transducer elements (e.g., approximately 85%) are unobstructed, and in this example conveys that information in the form of a gauge 775 along with color coding. In FIG. 7F, the simulated transducer array 759 has been moved to the side of the patient. As shown in the 2D view 771, more of the patient's ribs obstruct transducer elements compared to FIG. 7D, with the graph or meter 773 of FIG. 7G reflecting that more elements are blocked by showing a depleted gauge 775 and also a change from “green” to “yellow”. As described above, while color coding is one implementation, it should be understood that this information can be conveyed in a number of ways including by displaying a percentage or actual value or number of obstructed vs. unobstructed transducer elements. As can also be seen in FIG. 7F, the 2D view of the back projected rays can also be displayed on the simulated transducer array 759, as shown.

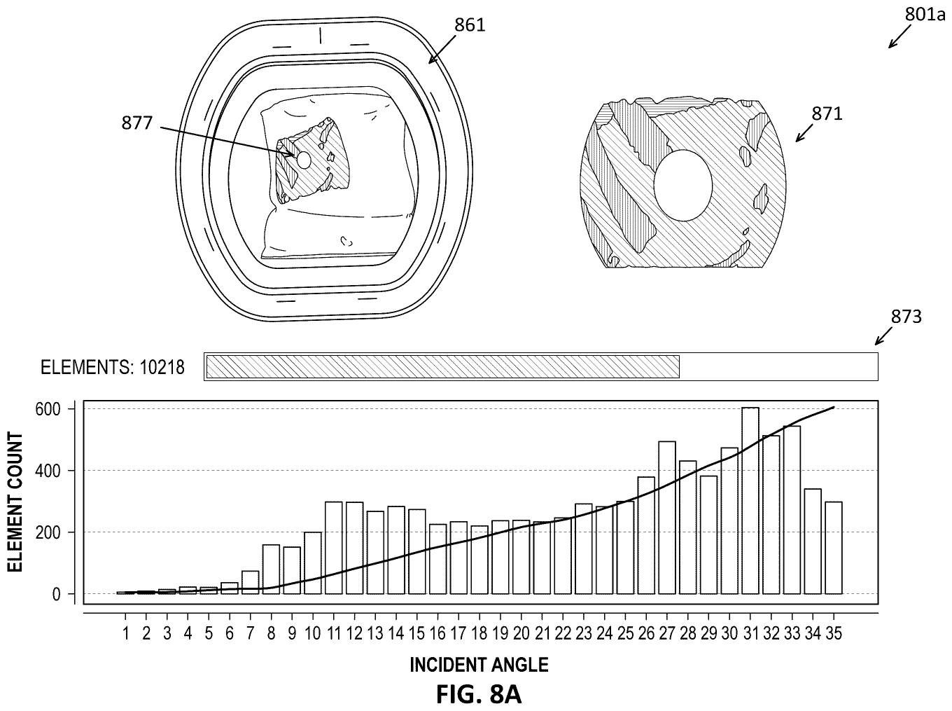

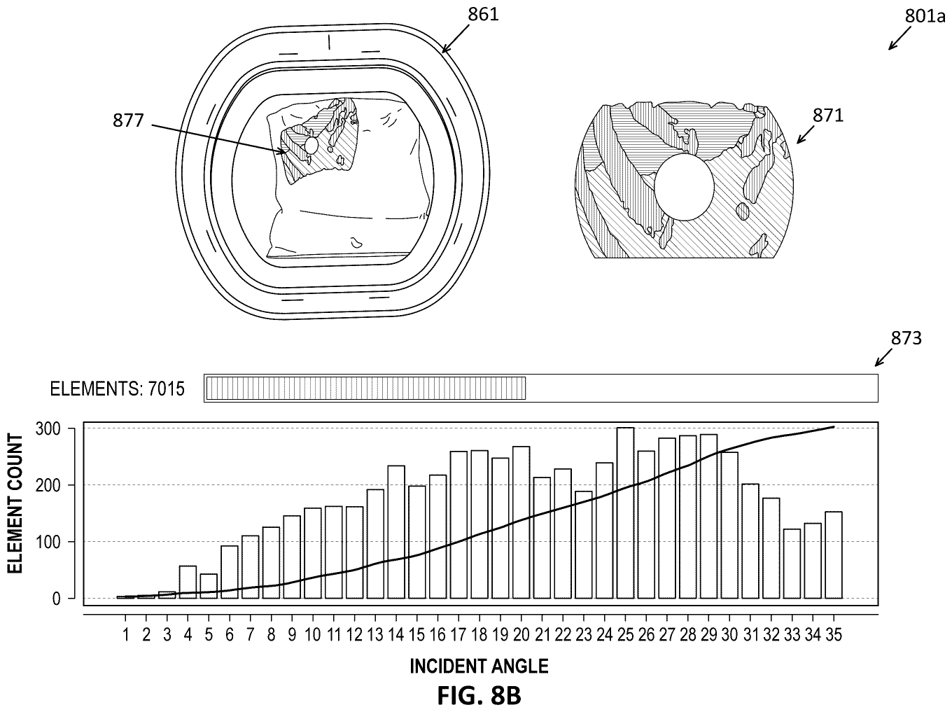

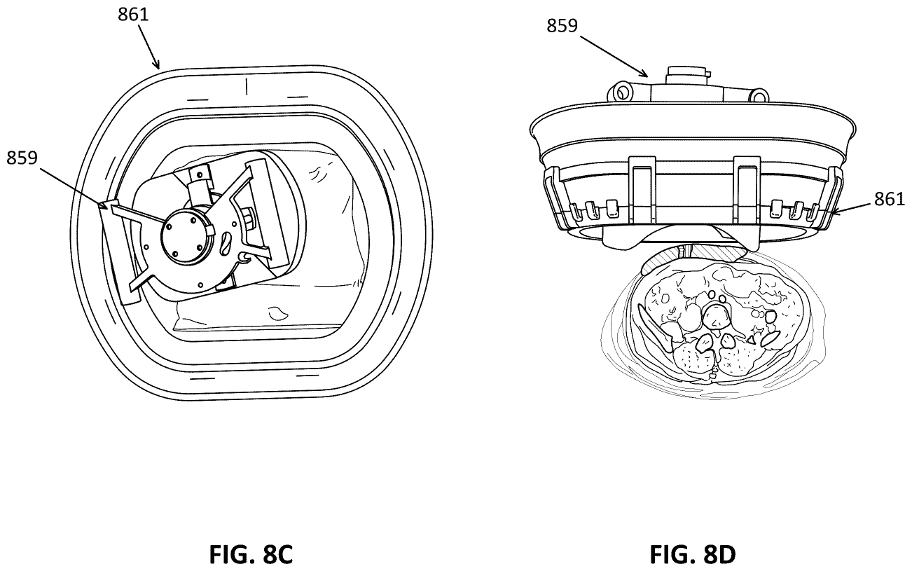

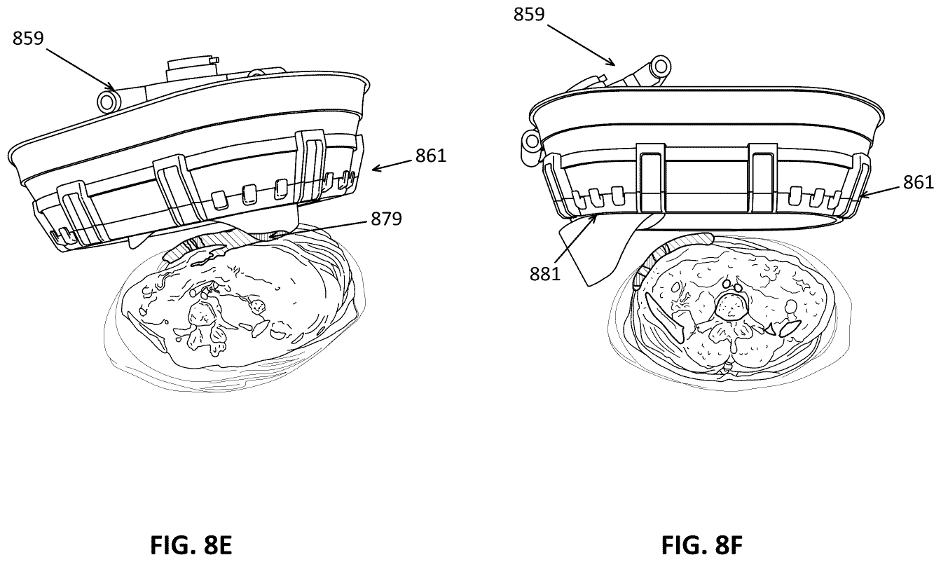

Referring to FIG. 8A, a UI screen 801a is shown with an intuitive view of the 2D view 871 of the back-projected rays can also be projected as a volume rendering 877 on a simulated model of the patient's skin by displaying the ray properties on the points where they cross the water/skin boundary. If the collisions with obstructing structures are displayed as different colors then the user can be provided with an interactive “X-ray” view of the anatomy to be avoided as the transducer pose is manipulated. Again, GPU implementation with the histotripsy system console makes this visualization smoothly interactive.

Software and corresponding UI displays can include both the simulated model of ray properties on a patient's skin or surface and a corresponding model of the obstructed, unobstructed and partially obstructed rays on the transducer face. As these two surfaces are displayed as mirror images of the various rays, as movement of the transducer is simulated across a patient, the various obstructed, unobstructed and partially obstructed rays will also change on the transducer face in addition to on the patient surface in the UI. As described herein, optimal transducer pose may be obtained (and recorded) by aligning the transducer and patient orientation for minimal acoustic obstruction. In this example, the acoustic coupling container 861 relative to the patient is also shown.

In combination with volume rendering of patient anatomy, in some embodiments the planning tool and/or histotripsy system can be configured to reveal or display underlying structures in the subject. Referring to FIGS. 8A-8B, an example of this skin projection technique is shown in combination with the 3D skin surface. The volume rendering 877 can be modulated such that soft tissue is clipped in combination with an oblique planar view of the source image data volume. Along with the 3D view of the skin surface obstruction projection, the simple 2D view 871 described above can also be displayed. In some embodiments, the volume rendering 877 can be displayed simultaneously with the 2D view 871 and the previously described graph or meter 873 for indicating the number or percentage of blocked transducer elements.

Alternatively, the user can select which view to display prior to or during a procedure. In combination with the interactive 3D presentation, the user receives interactive feedback that informs finding a transducer pose with a more optimal acoustic window to the target.

Additional features or components of the system can be optionally displayed to assist with treatment planning. For example, referring to FIGS. 8A-8B, the user can elect to display an acoustic coupling container 861 in place over the simulated patient (or patient imaging). In FIGS. 8C-8F, the user can elect to display the simulated transducer array 859. As described herein, the coupling container provides acoustic coupling between the transducer array and the patient and defines the volume or space within which the transducer array and robotic positioning arm can move during a given procedure. It can be useful to display this coupling container over a planned treatment volume to identify where the simulated transducer array can be moved, and to optionally identify more optimal patient poses or coupling container placements depending on the ray tracing results for a given treatment plan.

Additionally, the planning tool can be used to identify potential collisions between the various system components, either between each other (e.g., between robot/transducer and coupling container) or between a system component and the patient. For example, FIG. 8E shows an example of a simulated collision 879 between the transducer array 859 and the patient. This collision can be represented with colors or other visual indicators as described above. In some embodiments, the collision can be represented in terms of simulating how much force is being applied in the collision. For example, some degree of collision or force application from the transducer to the patient may be acceptable during a given therapy. The amount or degree of force that is applied can also be displayed with visual indicators as discussed herein (e.g., with red representing a significant force, yellow an acceptable force, and green no force or collision). Similarly, in FIG. 8F, the simulation or planning tool can show a collision 881 between the transducer array and the coupling container. In this example, it is shown as the transducer array clipping or passing through a boundary of the coupling container.

Using any of the techniques described above, the treatment planning tools of the present disclosure can provide an interactive ability for a user to digitally manipulate or modify the position/pose of a graphical representation of an ultrasound treatment head/transducer array in the context of a user-selected target within the patient. Manipulation of the position/pose of the virtual treatment head can allow the user to identify an optimal acoustic window into the target, since the treatment planning tool can provide real-time information relating to obstructions such as bone or gas between the treatment head and the target as the position/pose is modified. The treatment planning tool can provide visual indicators of obstructions or partial obstructions, allowing the user to identify a preferred position/pose for the target. The preferred position/pose of the treatment head can be stored digitally within the treatment plan, so that when the patient is on the treatment table during the actual therapeutic procedure, the treatment plan can be used to guide the physician to the preferred position/pose, or the treatment plan can automatically instruct the robotic positioning arm to place the treatment head in the preferred pose/position at the start of therapy without user intervention.

### Aberration Correction

Acoustic output of the transducer array reaches the geometric focal spot in a time determined by the path length of the ray from the transducer to the focus divided by the speed of sound along the path. In the case where several media, each with a different speed of sound, are traversed, the total time of flight is the sum of all the media path lengths divided by their respective speeds of sound. If the path lengths through a particular media vary across the face of the transducer, then the arrival times also vary. This leads to so called phase aberration and results in deconstructive interference. The result is typically a translated focal spot with reduced amplitude.

A transducer array composed of a sufficient number of independent sub transducer elements whose output can be independently controlled can potentially correct phase aberration by estimating a time delay for each element such that their signals reach the intended focal spot simultaneously. One technique for computing these delay values uses the ray tracing strategy described above. For each ray of the transducer array, the path lengths through the coupling medium and soft tissue together with estimated speeds of sound can be used to calculate the required time delay to reduce phase aberration. The effects of phase aberration can be reduced by estimating or simulating the time of flight from different transducer elements to the focus, and applying corresponding time delays on ultrasound emissions for appropriate transducer elements. This restores the peak to the geometric focus location and increases its amplitude.

In one embodiment, this can be implemented by using preoperative imaging fused with intraoperative ultrasound to bring the preoperative imaging into registration with the real world pose of the transducer. Ray tracing can then be used to estimate per element delay values from the preoperative imaging to reduce phase aberration. This correction could be used on its own or as an initialization to a subsequent measurement-based phase aberration correction technique. Efficient aberration correction increases the accuracy of treatment delivery and expands the treatment envelope.

### Pressure Simulation

Additionally, the planning tools provided herein are configured to perform pressure simulations for a given acoustic target location and/or target treatment volume within a 3D digital model of the subject to determine optimal drive voltages required to generate cavitation for a given treatment location, simulate and predict aberration correction algorithms that may be required as a result of obstructions or tissue changes. The treatment planning tools provided herein can optimize a given histotripsy therapy, including allowing for selection of an optimal acoustic window which may lower the cavitation initiation thresholds in a target tissue volume by up to 30-50% making the system have more efficient energy delivery.

Interactive pressure field simulations can be valuable for the predication of treatment feasibility and quality of a given target. Building upon the ray tracing methods discussed above, a rapid, GPU accelerated pressure simulation can be obtained by using the precomputed geometry of the traced rays to compute a Rayleigh-Sommerfeld integral over a 2D or 3D computational domain. In this way, the sum of the complex contributions of all the rays (typically several thousand) can be computed for each point in the target domain in parallel. On suitable current hardware this allows an interactive refresh rate of 5-10 Hz for a 2D field of 100×100 points considering approximately 15 thousand traced rays.

Computed 2D pressure fields can be shown in color along with magnitude plots and real signal value plots taken from the vertical midline of the 2D field. Both continuous wave and pulsed emissions of the transducer can be simulated by applying variable time and spatial windowing to the contributions of each traced ray to the result. This information can all be displayed to a user of the system during therapy.

In some aspects, the planning tools provided herein can use ray-tracing from a virtual histotripsy therapy transducer array into the 3D digital model of the patient to estimate one or more driving voltages of the virtual histotripsy therapy transducer array that would be required or necessary to produce cavitation within a target tissue volume for a given pose/position of the virtual histotripsy transducer array. Since the user is able to manipulate the position/pose of the virtual array, the treatment planning tool can repeat the pressure simulations until the user finds a position/pose of the array that provides efficient cavitation generation at acceptable voltage levels. The user can repeat moving the position/pose of the array and running the pressure simulations until a preferred pose/position of the array is found. The preferred pose/position, and the estimated driving voltages can be stored in a digital treatment plan for later use during therapy.

### Treatment Plan Quality Assessment

Fast pressure simulation capabilities allow for quantitative treatment plan quality metrics to be compiled interactively. Acoustic pressure created by the transducer typically varies across the extents of a prescribed treatment volume. As the transducer moves to place its focal point at different points within the treatment volume the beam path through intervening tissue changes accordingly, resulting in varying tissue acoustic properties which modulate the focal amplitude. These variations may result in parts of the prescribed treatment volume receiving focal pressure amplitudes below the threshold for bubble cloud formation, indicating that the transducer output must be increased for those portions of the volume. For power calibration adjustments of these kinds, manual user input is limited to surveying 7 points (center point plus extremes in each cardinal direction), with the user adjusting voltage to achieve cavitation. The user inputs are then interpolated across the treatment volume. Pressure simulation allows this process to be automated, with more thorough sampling of the volume made feasible.

Additionally, treatment plan quality can be interactively conveyed to the user through something analogous to dose volume histograms of target (and surrounding spare volumes of interest if desired). Volume rendered treatment quality maps can also be created to give 3D context to the planned volumes.

Therefore, in one aspect, a pre-treatment planning technique can be used in which pressure simulations are used to determine pressure thresholds throughout a target tissue volume. When a treatment plan is generated for the target tissue volume and populated with a plurality of treatment locations, the pressure simulations can be used to determine the cavitation threshold and/or driving voltages/amplitudes for the transducer array for some or all of the plurality of treatment locations. Additionally, the pressure simulation across a prescribed treatment volume can be used to estimate total treatment time, taking into account an estimation of body wall heating and accounting for any required cooling time during therapy. Therapy can then proceed with the proposed treatment plan according to the driving voltages as determined by the pressure simulation.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of planning histotripsy therapy, comprising:
obtaining at least one diagnostic image of a subject including a target tissue location;
generating a three-dimensional (3D) digital model of the subject and the target tissue location from the at least one diagnostic image;
positioning a virtual histotripsy therapy transducer array near the 3D model with a geometric focus of the virtual histotripsy therapy transducer positioned on or near the target tissue location in the 3D digital model;
sampling the virtual histotripsy therapy transducer array to provide starting points for a plurality of rays that correspond to simulated ultrasound energy delivered by transducer elements of the virtual histotripsy therapy transducer;
tracing the plurality of rays through the 3D digital model; and
identifying one or more obstructed or partially obstructed transducer elements of the virtual histotripsy therapy transducer array corresponding to rays that pass through an obstruction in the 3D digital model.

2. The method of claim 1, further comprising displaying the rays that pass through an obstruction to a user.

3. The method of claim 1, further comprising providing a first graphical representation of the rays that pass through an obstruction and providing a second graphical representation of the rays that do not pass through an obstruction.

4. The method of claim 3, wherein rays that pass through an obstruction are displayed in a first color and rays that do not pass through an obstruction are displayed in a second color.

5. The method of claim 1, wherein the 3D digital model further comprises a skin surface model of the subject.

6. The method of claim 5, further comprising presenting a map of rays that pass through an obstruction on the skin surface model.

7. The method of claim 2, wherein displaying the rays that pass through an obstruction further comprises providing a 2D view of the plurality of rays.

8. The method of claim 1, further comprising:
receiving an input to adjust a position or pose of the virtual histotripsy therapy transducer;
re-sampling the virtual histotripsy therapy transducer array to provide starting points for a second plurality of rays that correspond to simulated ultrasound energy delivered by transducer elements of the virtual histotripsy therapy transducer array;
tracing the second plurality of rays through the 3D digital model; and
identifying one or more obstructed or partially obstructed transducer elements of the virtual histotripsy therapy transducer array corresponding to rays that pass through an obstruction in the 3D digital model.

9. The method of claim 1, further comprising generating a digital treatment plan that includes the target tissue location and a pose or position of the virtual histotripsy therapy transducer array.

10. The method of claim 1, wherein the digital treatment plan further includes a depth and size of the target tissue location.

11. The method of claim 1, further comprising segmenting one or more target tissues or organs in proximity to the target tissue location.

12. A method of planning histotripsy therapy, comprising:
obtaining at least one diagnostic image of a subject including a target tissue location;
generating a three-dimensional (3D) digital model of the subject and the target tissue location from the at least one diagnostic image;
positioning a virtual histotripsy therapy transducer array near the subject with a geometric focus of the histotripsy therapy transducer array positioned on or near the target tissue location;
sampling the histotripsy therapy transducer to provide starting points for a plurality of rays that correspond to ultrasound energy delivered by transducer elements of the histotripsy therapy transducer array;
tracing the plurality of rays through the 3D digital model;
generating a pressure simulation within 3D digital model based on the plurality of rays; and
estimating one or more driving voltages of the virtual histotripsy therapy transducer array required to generate cavitation at the target tissue location based on the pressure simulation.

13. The method of claim 12, wherein generating the pressure simulation further comprises computing a Rayleigh-Sommerfeld integral over a 2D or 3D computational domain.

14. The method of claim 12, further comprising displaying rays that pass through an obstruction to a user.

15. The method of claim 12, further comprising providing a first graphical representation of rays that pass through an obstruction and providing a second graphical representation of rays that do not pass through an obstruction.

16. The method of claim 15 wherein rays that pass through an obstruction are displayed in a first color and rays that do not pass through an obstruction are displayed in a second color.

17. The method of claim 12, wherein the 3D digital model further comprises a skin surface model of the subject.

18. The method of claim 17, further comprising presenting a map of rays that pass through an obstruction on the skin surface model.

19. The method of claim 12, further comprising:
receiving an input to adjust a position or pose of the virtual histotripsy therapy transducer;
re-sampling the virtual histotripsy therapy transducer array to provide starting points for a second plurality of rays that correspond to simulated ultrasound energy delivered by transducer elements of the virtual histotripsy therapy transducer array;
tracing the second plurality of rays through the 3D digital model; and
identifying one or more obstructed or partially obstructed transducer elements of the virtual histotripsy therapy transducer array corresponding to rays that pass through an obstruction in the 3D digital model.

20. A histotripsy treatment planning tool, comprising:
a display;
a user interface;
one or more processors operatively coupled to the display and the user interface;
a non-transitory computing device readable medium having instructions stored thereon for generating a treatment plan for ultrasound therapy, wherein the instructions are executable by the one or more processors to:
receive one or more diagnostic images of a subject including a target tissue location;
present a three-dimensional (3D) model of the subject and the target tissue location on the display;
present a virtual ultrasound therapy transducer array in proximity to the 3D model of the subject;
receive an input from the user-interface that includes instructions to modify a pose or position of the virtual ultrasound therapy transducer array;
simulate a plurality of rays from the virtual ultrasound therapy transducer array for a given pose and position into the 3D model of the subject and into the target tissue location; and
present information on the display indicating if any of the plurality of rays pass through an obstruction within the 3D model of the subject.
